(19) United States
(12) Patent Application Publication
(10) Pub. No.: US 2024/0327932 A1
(43) Pub. Date: Oct. 3, 2024

(54) BIOMARKER-BASED METHOD AND SYSTEM FOR IDENTIFYING COMPANION ANIMALS HAVING RISK FOR GASTROINTESTINAL PROBLEMS

(71) Applicant: Hill's Pet Nutrition, Inc., Overland Park, KS (US)

(72) Inventors: Dayakar BADRI, Lawrence, KS (US); Christina GOLDER, Topeka, KS (US); Chun-Yen COCHRANE, Manhattan, KS (US)

(73) Assignee: Hill's Pet Nutrition, Inc., Overland Park, KS (US)

(21) Appl. No.: 18/622,125

(22) Filed: Mar. 29, 2024

Related U.S. Application Data
(60) Provisional application No. 63/493,128, filed on Mar. 30, 2023.

(51) Int. Cl. C12Q 1/689; G16H 20/60; G16H 50/30

(52) U.S. Cl. CPC C12Q 1/689; G16H 20/60; G16H 50/30; C12Q 2600/118

(57) ABSTRACT

Described herein are methods for identifying companion animals at a greater risk for developing a gastrointestinal (GI) disease/condition. In particular, described herein are holistic comprising the use of gut metabolites (SCFA), physiological state (pH) and fecal scores (1-5) to identify a companion animal at a greater risk for developing a GI condition/disease. Compositions, methods and kits for treating companion animals who have been so identified, are also described.

### CROSS REFERENCE TO RELATED APPLICATION

This application claims the benefit of U.S. provisional patent application 63/493,128, which was filed Mar. 30, 2023, is titled Biomarker-Based Method and System for Identifying Companion Animals Having Risk for Gastrointestinal Problems, and is incorporated herein by reference as if fully set forth.

### BACKGROUND

The accurate classification of individual dogs having a risk or no risk of gastrointestinal (GI) problems as “GI risk” vs “No GI risk” remains a significant clinical challenge, particularly using non-invasive sampling (e.g., sampling of feces).

The gut microbiome performs numerous important biochemical functions by forming mutualistic symbiotic associations to the host. Perturbation of the microbiome causes dysbiosis, which is positively associated with many non-communicable diseases and conditions (NCDs). For example, dysbiosis is positively associated with chronic kidney disease, cardiovascular disease, circulatory disease, inflammatory bowel disease (IBD), chronic enteropathy, chronic kidney disease (CKD), autism, celiac disease, Crohn's disease, ulcerative colitis, prostate cancer, colorectal cancer, obesity, type 1 and 2 diabetes type, and some skin diseases. Dysbiosis can also affect the gut-brain axis, the gut-brain-skin axis, and drug metabolism and toxicity therefrom. Dysbiosis also influence the host immune response.

Predominant studies demonstrated that “dysbiosis” is a condition that causes most of the host pathological states mentioned above. Recent evidence supported the development of a “dysbiosis index” based on the abundance of specific microbial species in the gut to predict the host risk condition for specific diseases (e.g., IBD & chronic enteropathy) in humans and dogs. Besides the microbiome, gut metabolism and physiology via the microbiome is also a critical factor in the development/or maintenance and onset of certain disease states. Evidence has clearly demonstrated that certain metabolites, particularly Short-Chain Fatty Acids (SCFA) and pH along with microbiome are involved in colorectal cancer onset and suggests that SCFA and pH are contributors to the state of “dysbiosis.”

Three important factors: (1) the microbiome, (2) gut metabolism, and (3) physiology are critical for the development of GI maintenance versus dysbiosis and onset of certain disease states. However, the dysbiosis index developed by these studies is of limited application because it is completely dependent only upon the abundance of particular bacterial species.

There is a need, therefore, for more comprehensive approaches that take into account multiple parameters, including microbiome, gut metabolites (e.g., SCFA), physiological state (pH), and fecal scores (1-5, wherein 1=liquid/watery and 5=firm and cylindrical in shape), to develop a model to identify if an individual dog has a risk or is not at risk of developing GI problems. Generally, a fecal score of 4-5 is desirable.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The invention relates to identifying dogs having or not having a risk for GI problems.

In an embodiment, the disclosure relates to a method for identifying a subject dog as being at risk or not at risk for gastrointestinal (GI) disease. The method comprises (a) analyzing a fecal sample from the subject dog to determine fecal sample values for one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes. The method also comprises (b) comparing the values obtained from (a) with control values from one or more not at risk for GI disease dog for a respective one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes. The subject dog is not at risk for GI disease when one or more of the fecal sample values are similar or equal to the control values. In some embodiments, the subject dog is not at risk for GI disease when each of the fecal sample values are similar or equal to the control values. In some embodiments, the subject dog is at risk for GI disease when one or more of the fecal sample values is different than the control values. In some embodiments, the subject dog is at risk for GI disease when each of the fecal sample values is different than the control values.

In an embodiment, the disclosure relates to a kit for identifying a subject dog as being at risk or not at risk for gastrointestinal (GI) disease. The kit comprises at least one of: a reagent or device for determining pH of a fecal sample, one or more reagent or device for measuring volatile carboxylic acids in a fecal sample, and one or more reagent or device for determining an abundance in a fecal sample of one or more of abundance of bacteria of Prevotellaceae, Ruminococcaceae, Bacteroidaceae, Enterobacteriaceae, and Bacteroidetes.

In an embodiment, the disclosure relates to a method for preventing or treating GI disease comprising identifying whether a subject dog is at risk or not at risk for gastrointestinal (GI) disease, and providing to the subject dog a GI disease-mitigating diet when the identifying step is a conclusion that the subject dog is at risk. In some embodiments, the step of identifying a subject dog as being at risk or not at risk for gastrointestinal (GI) disease comprises: (a) analyzing a fecal sample from the subject dog to determine fecal sample values for one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes; and (b) comparing the values obtained from (a) with control values from one or more not at risk for GI disease dog for a respective one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes. The subject dog is not at risk for GI disease when one or more of the fecal sample values is similar or equal to the control values, or wherein the subject dog is at risk of GI disease when on or more of the fecal sample values is different than the control values. In some embodiments, the GI disease-mitigating diet comprises a dog food composition quinoa, amaranth, egg shell membrane, and dietary fiber. In some embodiments, the dog food composition comprises from about 1 to about 10 wt. % of quinoa, from about 1 to about 10 wt. % of amaranth; from about 0.5 to about 8 wt. % of egg shell membrane, and from about 2 to about 20 wt. % of dietary fiber. In some embodiments, the dietary fiber comprises insoluble fiber and soluble fiber. In some embodiments, the dietary fiber comprises from about 1 to about 18 wt. % of insoluble fiber, and from about 0.5 to about 8 wt. % of soluble fiber. The weight percentages are based on the total weight of the dog food composition on a dry matter basis.

In an embodiment, the disclosure relates to a device comprising an input, a memory, a processor, and an output. The input is configured to receive the results of a method for identifying a subject dog as being at risk or not at risk for gastrointestinal (GI) disease. The memory comprises processor executable instructions for comparing the results to control values, for preparing a conclusion of at risk or not at risk based on the comparing, and for outputting the results. The processor is configured to execute the processor executable instructions. The output configured to display the conclusions. In some embodiments, the processor executable instructions further comprise instructions to display a recommendation to feed to the subject a GI disease-mitigating diet when conclusion is that the subject dog is at risk, the processor is further configured to execute the instructions to display a recommendation, and the output is further configured to display the recommendation.

In an embodiment, provided is a method for identifying a subject dog as being at risk or not at risk for gastrointestinal (GI) disease comprising: (a) analyzing a feces sample from the dog for pH, total short chain fatty acid content (SCFA), and three or more of: C2 SCFA, C3 SFCA, C4 SCFA, C5 SCFA, and/or C6 SCFA content, feces score, moisture, and relative abundance of Prevotellaceae, Phylum Bacteroidetes-excluding F3: f_Bacteroidaceae and F4: f__Prevotellaceae, Ruminococcaceae, Enterobacteriaceae, Clostridiales and Prevotella; and optionally other bacterial phyla, families, genera or species. The method also comprises (b) comparing the values obtained from (a) with control values obtained from one dog or pooled from a plurality of dogs that have been medically determined to not be at risk for GI disease. The subject dog is identified as not being at risk for GI disease when the feces sample has: (i) approximately equal or lower pH and approximately equal or higher levels of total SFCA than the control values, and (ii) when measured, approximately equal or lower values for moisture and/or Enterobacteriaceae than the control values, and approximately equal or higher levels of C3 SCFA, C2 SFCA, C4 SCFA, feces score, Prevotellaceae, Ruminococcaceae, Clostridiales and/or Prevotella than the control values. The presence of other bacteria may be determined because greater diversity in the gut microbiome is also generally associated with No Risk dogs.

In an embodiment, provided is a kit for identifying a subject dog as being at risk or not at risk for gastrointestinal (GI) disease. The kit comprises a reagent for determining pH of a fecal sample, one or more reagents for measuring volatile carboxylic acids in the fecal sample, and one or more reagents for quantitatively identifying in a fecal sample one or more of Prevotellaceae, Phylum Bacteroidetes, excluding F3: f_Bacteroidaceae and F4: f_Prevotellaceae, Ruminococcaceae, Enterobacteriaceae, Clostridiales, and/or Prevotella. In some embodiments, the method further comprises quantitatively identifying other bacteria.

In an embodiment, provided is a method for preventing or treating GI disease comprising identifying GI status of dog and providing to a dog identified as at risk for GI disease with a GI disease-mitigating diet, as further described herein.

### BRIEF DESCRIPTION OF THE DRAWINGS

The features, and advantages of the invention will be apparent from the following more detailed description of certain embodiments of the invention and as illustrated in the accompanying drawings in which:

FIGS. 1A and 1B show principal component analysis of 96 variables derived from pH, SCFA, microbiome and other metadata.

FIG. 2 shows Receiving Operating Characteristics (ROC) of the discovery cohort and associated confusion matrix for prediction.

FIGS. 3A and 3B show abundance of important variables used in the model to discriminate individuals into two groups (GI risk vs No GI risk). Microbiome variables were represented as relative abundance (Center Log Ratio transformed) and other variables were represented by actual levels. SCFAs are represented in ppm and moisture is represented in %.

FIGS. 4A and 4B show principal component analysis (PCA) using selected discriminators to distinguish No GI Risk (open diamonds) from GI Risk (closed circles).

FIGS. 5A and 5B show scatter plots of the important variables with significant correlation to pH. Open diamonds=No GI Risk; Closed circles=GI Risk. Microbiome variables were represented as relative abundance and other variables were represented by actual levels.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to and can be employed in other compositions and methods. Before explaining the disclosed embodiments of the present invention in detail, it is to be understood that the invention is not necessarily limited in its application to the details of any particular embodiment disclosed. The terminology used herein is for the purpose of description and not of limitation.

As used herein and in the appended claims, the singular forms “a,” “an,” and “the” include plural references unless the context dictates otherwise. The singular form of any class of the ingredients refers not only to one ingredient within that class, but also to a mixture of those ingredients. The terms “a” (or “an”), “one or more” and “at least one” may be used interchangeably herein. The terms “comprising,” “including,” and “having” may be used interchangeably. A list phrased as “at least one of A, B, and C” or “at least one of A, B, or C” means any one or more of A, B, and C, rather than at least one A, at least one B, and/or at least one C. The term “include” should be interpreted as “include but are not limited to”. The term “including” should be interpreted as “including but are not limited to” or “including but is not limited to.”

As used throughout, ranges are used as shorthand for describing each and every value that is within the range. Any value within the range can be selected as the terminus of the range. Thus, a range from 1-5, includes specifically 1, 2, 3, 4 and 5, as well as subranges including 1-4, 1-3, 1-2, 2-5, 2-4, 2-3, 3-5, 3-4, and 4-5. The term “about” when referring to a number means any number within a range of 15% of the number. Further embodiments herein include removing the word “about” at one or more instance in an embodiment including “about” herein. Further embodiments herein include removing the word “about” at every instance in an embodiment including “about” herein.

The abbreviations and symbols as used herein, unless indicated otherwise, take their ordinary meaning. The abbreviation “wt. %” means percent by weight with respect to a pet food composition. The symbol “°” refers to a degree, such as a temperature degree or a degree of an angle. The symbols “h”, “min”, “mL”, “nm”, “μm” means hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

All references cited herein are hereby incorporated by reference in their entireties. In the event of a conflict in a definition in the present disclosure and that of a cited reference, the present disclosure controls.

An embodiment comprises a method for identifying a subject dog as being at risk or not at risk for gastrointestinal (GI) disease. The method comprises (a) analyzing a fecal sample from the subject dog to determine fecal sample values for one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes. The method also comprises (b) comparing the values obtained from (a) with control values from one or more not at risk for GI disease dog for a respective one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes. The control values may be obtained from reference sources or tables of control values previously obtained. The control values may be from this specification. The control values may be obtained from experiments on one or more not at risk for GI disease dog conducted using the same methods for one or more of analyzing a fecal sample from the subject dog to determine fecal sample values for one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes, but conducting the analysis on fecal sample(s) from the control dog(s).

In some embodiments, the subject dog is determined to be not at risk for GI disease when one or more of the fecal sample values are similar or equal to the control values.

In some embodiments, the step (a) of analyzing is conducted to determine the fecal sample values for each of the (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes. In some embodiments, the (b) comparing is with the control values for each respective one of the (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes.

In some embodiments, the subject dog is determined not at risk for GI disease when each of the fecal sample values are similar or equal to the control values. In some embodiments, the subject dog is determined to be at risk for GI disease when each of the fecal sample values is different than the control values.

In some embodiments, he subject dog is determined to be not at risk for GI disease when the fecal sample has, compared to the control values: (i) an approximately equal to or higher level total short chain fatty acid content; (ii) an approximately equal to or higher level of C2 SCFA; (iii) an approximately equal to or higher level of C3 SCFA; (iv) an approximately equal to or higher level of C4 SCFA; (v) an approximately equal to or lower pH; (vi) an approximately equal to or lower moisture, (vii) an approximately equal to or higher fecal score; and (viii) an approximately equal to or higher abundance of bacteria of Prevotellaceae, Ruminococcaceae, and Bacteroidaceae, and an approximately equal to or lower abundance of bacteria of Enterobacteriaceae and Bacteriodetes other than Prevotellaceae and Bacteroidaceae.

In some embodiments, the subject dog is determined to be at risk for GI disease when the feces sample has, compared to the control values: (i) a lower level of total short chain fatty acid content; (ii) a lower level of C2 SCFA; (iii) a lower level of C3 SCFA; (iv) a lower level of C4 SCFA; (v) a higher pH; (vi) a higher moisture; (vii) a lower fecal score; and (viii) a lower abundance of bacteria of Prevotellaceae, Ruminococcaceae, and Bacteroidaceae, and an approximately higher abundance of bacteria of Enterobacteriaceae and Bacteriodetes other than Prevotellaceae and Bacteroidaceae.

In some embodiments, the subject dog is determined to be not at risk for GI disease when the feces sample has, compared to the control values, at least one of, at least two of, at least three of, at least four of, at least five of, at least six of, at least seven, or at least eight of: (i) an approximately equal to or higher levels total short chain fatty acid content; (ii) an approximately equal to or higher level of C2 SCFA; (iii) an approximately equal to or higher level of C3 SCFA; (iv) an approximately equal to or higher level of C4 SCFA; (v) an approximately equal to or lower pH; (vi) an approximately equal to or lower moisture, (vi) an approximately equal to or higher fecal score; or (viii) one or both of equal to or higher abundance of bacteria of one or more of Prevotellaceae, Ruminococcaceae, and Bacteroidaceae, and an approximately equal to or lower abundance of bacteria of one or both of Enterobacteriaceae and Bacteriodetes other than Prevotellaceae and Bacteroidaceae.

In some embodiments, the subject dog is determined to be at risk for GI disease when the feces sample has, compared to the control values, at least one, two, three four, five, six, or seven of: (i) a lower level total short chain fatty acid content; (ii) a lower level of C2 SCFA; (iii) a lower level of C3 SCFA; (iv) a lower level of C4 SCFA; (v) a higher pH; (vi) higher moisture, (vii) a lower fecal score; or (viii) one or both of a lower abundance of bacteria of one or more of Prevotellaceae, Ruminococcaceae, and Bacteroidaceae, and an approximately higher abundance of bacteria of one or both of Enterobacteriaceae and Bacteriodetes other than Prevotellaceae and Bacteroidaceae.

An embodiment comprises a method for identifying a subject dog as being at risk or not at risk for gastrointestinal (GI) disease. The method comprises (a) analyzing a fecal sample from the subject dog to determine fecal sample values for one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes. The subject dog is not at risk for GI disease when the fecal sample values includes one or more of: (i) the total short chain fatty acid content (SCFA) as found for not at risk dogs in examples herein; (ii) the level of C2 SCFA as found for not at risk dogs in examples herein; (iii) the level of C3 SCFA as found for not at risk dogs in examples herein; (iv) the level of C4 SCFA as found for not at risk dogs in examples herein; (v) the pH as found for not at risk dogs in examples herein; (vi) the moisture as found for not at risk dogs in examples herein; (vii) the fecal score as found for not at risk dogs in examples herein; and (vii) an abundance of bacteria of one or more of Prevotellaceae, Ruminococcaceae, Bacteroidaceae, Enterobacteriaceae, and Bacteroidetes as found for not at risk dogs in examples herein. In some embodiments, the fecal sample values include each of (i) through (vii). In some embodiments, the fecal sample values include each of (i) through (vii) and the values for (viii) include each of an abundance of bacteria of Prevotellaceae, Ruminococcaceae, Bacteroidaceae, Enterobacteriaceae, and Bacteroidetes.

In some embodiments, the subject dog is determined to be at risk for GI disease when the fecal sample values include one or more of: (i) the total short chain fatty acid content (SCFA) less than that observed for not at risk dogs in examples herein; (ii) the level of C2 SCFA less than that observed for not at risk dogs in examples herein; (iii) the level of C3 SCFA less than that observed for not at risk dogs in examples herein; (iv) the level of C4 SCFA less than that observed for not at risk dogs in examples herein; (v) the pH greater than that observed for not at risk dogs in examples herein; (vi) the moisture greater than that observed for not at risk dogs in examples herein; (vii) the fecal score less than that observed for not at risk dogs in examples herein; and (viii) one or more of an abundance of bacteria of one or more of Prevotellaceae, Ruminococcaceae, and Bacteroidaceae less than that observed for not at risk dogs in examples herein, and one or both of an abundance of bacteria of Enterobacteriaceae and Bacteriodetes, other than Prevotellaceae and Bacteroidaceae, greater than that observed for not at risk dogs in examples herein. In some embodiments, the fecal sample values include each of (i) through (vii). In some embodiments, the fecal sample values include each of (i) through (vii), and the values for (viii) include each of an abundance of bacteria of Prevotellaceae, Ruminococcaceae, and Bacteroidaceae less than that observed for not at risk dogs in examples herein and an abundance of each of bacteria of Enterobacteriaceae and Bacteriodete greater than that observed for not at risk dogs in examples herein.

In some of the embodiments, the method of determining any of (i) through (vii) and any one of bacteria of Prevotellaceae, Ruminococcaceae, Bacteroidaceae, Enterobacteriaceae, and Bacteroidetes may be as set forth in an example herein.

An embodiment comprises a kit for identifying a subject dog as being at risk or not at risk for gastrointestinal (GI) disease by obtaining fecal analysis results. The kit comprises at least one of: a reagent or device for determining pH of a fecal sample, one or more reagent or device for measuring volatile carboxylic acids in a fecal sample, and one or more reagent or device for determining an abundance in a fecal sample of bacteria of one or more of Prevotellaceae, Ruminococcaceae, Bacteroidaceae, Enterobacteriaceae, and Bacteroidetes. Implementing the foregoing components of the kit produces the fecal analysis results.

In some embodiments, the kit comprises each of the reagent or device for determining pH of a fecal sample, the one or more reagent or device for measuring volatile carboxylic acids in a fecal sample, and the one or more reagent or device for determining an abundance in a fecal sample for each of Bacteriodetes bacteria, Prevotellaceae bacteria, Ruminococcaceae bacteria, Enterobacteriaceae bacteria, Bacteroidaceae bacteria, and Prevotella bacteria.

In some embodiments, the kit comprises the one or more reagent for determining the abundance of Enterobacteriaceae and Prevotellaceae in the sample.

In some embodiments, the one or more reagent or device for determining an abundance in a fecal sample for one or more of Bacteriodetes bacteria, Prevotellaceae bacteria, Ruminococcaceae bacteria, Enterobacteriaceae bacteria, Bacteroidaceae bacteria, and Prevotella bacteria comprise PCR primers for amplifying 16S rDNA from the target bacteria.

In some embodiments, the kit further comprises instructions for analyzing results of analysis of a subject dog fecal sample with the kit. In some embodiments, the instructions for analyzing comprise concluding the subject dog is at risk of for gastrointestinal (GI) disease when the fecal sample values include one or more of: (i) the total short chain fatty acid content (SCFA) less than that observed for not at risk dogs in examples herein; (ii) the level of C2 SCFA less than that observed for not at risk dogs in examples herein; (iii) the level of C3 SCFA less than that observed for not at risk dogs in examples herein; (iv) the level of C4 SCFA less than that observed for not at risk dogs in examples herein; (v) the pH greater than that observed for not at risk dogs in examples herein; (vi) the moisture greater than that observed for not at risk dogs in examples herein; (vii) the fecal score less than that observed for not at risk dogs in examples herein; and (viii) one or more of an abundance of bacteria of one or more of Prevotellaceae, Ruminococcaceae, and Bacteroidaceae less than that observed for not at risk dogs in examples herein, and one or both of an abundance of bacteria of Enterobacteriaceae and Bacteriodetes, other than Prevotellaceae and Bacteroidaceae, greater than that observed for not at risk dogs in examples herein. In some embodiments, the instructions for analyzing comprises concluding the subject dog is at risk of for gastrointestinal (GI) disease when the fecal sample values include each of: (i) the total short chain fatty acid content (SCFA) less than that observed for not at risk dogs in examples herein; (ii) the level of C2 SCFA less than that observed for not at risk dogs in examples herein; (iii) the level of C3 SCFA less than that observed for not at risk dogs in examples herein; (iv) the level of C4 SCFA less than that observed for not at risk dogs in examples herein; (v) the pH greater than that observed for not at risk dogs in examples herein; (vi) the moisture greater than that observed for not at risk dogs in examples herein; (vii) the fecal score less than that observed for not at risk dogs in examples herein; and (viii) one or more of an abundance of bacteria of one or more of Prevotellaceae, Ruminococcaceae, and Bacteroidaceae less than that observed for not at risk dogs in examples herein, and one or both of an abundance of bacteria of Enterobacteriaceae and Bacteriodetes, other than Prevotellaceae and Bacteroidaceae, greater than that observed for not at risk dogs in examples herein. In some embodiments, the instructions for analyzing comprise concluding the subject dog is not at risk of for gastrointestinal (GI) disease when the fecal sample values include one or more of: (i) the total short chain fatty acid content (SCFA) as found for not at risk dogs in examples herein; (ii) the level of C2 SCFA as found for not at risk dogs in examples herein; (iii) the level of C3 SCFA as found for not at risk dogs in examples herein; (iv) the level of C4 SCFA as found for not at risk dogs in examples herein; (v) the pH as found for not at risk dogs in examples herein; (vi) the moisture as found for not at risk dogs in examples herein; (vii) the fecal score as found for not at risk dogs in examples herein; and (viii) an abundance of bacteria of one or more of Prevotellaceae, Ruminococcaceae, Bacteroidaceae, Enterobacteriaceae, and Bacteroidetes as found for not at risk dogs in examples herein. In some embodiments, the instructions for analyzing comprises concluding the subject dog is not at risk of for gastrointestinal (GI) disease when the fecal sample values include each of: (i) the total short chain fatty acid content (SCFA) as found for not at risk dogs in examples herein; (ii) the level of C2 SCFA as found for not at risk dogs in examples herein; (iii) the level of C3 SCFA as found for not at risk dogs in examples herein; (iv) the level of C4 SCFA as found for not at risk dogs in examples herein; (v) the pH as found for not at risk dogs in examples herein; (vi) the moisture as found for not at risk dogs in examples herein; (vii) the fecal score as found for not at risk dogs in examples herein; and (vii) an abundance of bacteria of one or more of Prevotellaceae, Ruminococcaceae, Bacteroidaceae, Enterobacteriaceae, and Bacteroidetes as found for not at risk dogs in examples herein.

In some embodiments, the instructions outline a method for identifying a subject dog as being at risk or not at risk for gastrointestinal (GI) disease through implementing elements of the kit. The method comprises (a) analyzing a fecal sample from the subject dog to determine fecal sample values for one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (vii) abundance of bacteria of Prevotellaceae, Ruminococcaceae, Bacteroidaceae, Enterobacteriaceae, and Bacteroidetes. The method also comprises (b) comparing the values obtained from (a) with control values from one or more not at risk for GI disease dog for a respective one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (vii) abundance of bacteria of Prevotellaceae, Ruminococcaceae, Bacteroidaceae, Enterobacteriaceae, and Bacteroidetes. The control values may be obtained from reference sources or tables of control values previously obtained. The control values may be from this specification. The control values may be obtained from experiments on one or more not at risk for GI disease dog conducted using the same methods for one or more of analyzing a fecal sample from the subject dog to determine fecal sample values for one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (vii) abundance of bacteria of Prevotellaceae, Ruminococcaceae, Bacteroidaceae, Enterobacteriaceae, and Bacteroidetes, but conducting the analysis on fecal sample(s) from the control dog(s).

In some embodiments, the instructions outline a method for identifying a subject dog as being at risk or not at risk for gastrointestinal (GI) disease through implementing elements of the kit. The method comprises (a) analyzing a fecal sample from the subject dog to determine fecal sample values for one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (vii) abundance of bacteria of Prevotellaceae, Ruminococcaceae, Bacteroidaceae, Enterobacteriaceae, and Bacteroidetes. The subject dog is not at risk for GI disease when the fecal sample values includes one or more of: (i) the total short chain fatty acid content (SCFA) as found for not at risk dogs in examples herein; (ii) the level of C2 SCFA as found for not at risk dogs in examples herein; (iii) the level of C3 SCFA as found for not at risk dogs in examples herein; (iv) the level of C4 SCFA as found for not at risk dogs in examples herein; (v) the pH as found for not at risk dogs in examples herein; (vi) the moisture as found for not at risk dogs in examples herein; (vii) the fecal score as found for not at risk dogs in examples herein; and (viii) an abundance of bacteria of one or more of Prevotellaceae, Ruminococcaceae, Bacteroidaceae, Enterobacteriaceae, and Bacteroidetes as found for not at risk dogs in examples herein. In some embodiments, the fecal sample values include each of (i) through (viii). In some embodiments, the fecal sample values include each of (i) through (vii), and the values for (viii) include each of an abundance of Prevotellaceae, Ruminococcaceae, Bacteroidaceae, Enterobacteriaceae, and Bacteroidetes as found for not at risk dogs in examples herein.

In some embodiments, the kit further comprises a device comprising, an input, a memory, a process, and an output. The input is configured to receive the results of any method herein. In some embodiments, the input is configured to receive the results of a method conducted while implementing the other components of the kit. The memory comprises processor executable instructions for comparing the results to control values, and for preparing a conclusion of at risk or not at risk based on the comparing, an outputting the results. The processor is configured to execute the processor executable instructions. The output is configured to display the conclusions. In some embodiments, the processor executable instructions further comprise instructions to display a recommendation to feed the subject do a GI disease-mitigating diet when the conclusion is that the subject dog is at risk, and the output is further configured to display the recommendation.

An embodiment comprises a method for preventing or treating GI disease. The method comprises identifying whether a subject dog is at risk or not at risk for gastrointestinal (GI) disease. The method also comprises providing to the subject dog a GI disease-mitigating diet when the identifying step is a conclusion that the subject dog is at risk. The method of identifying whether a subject dog is at risk or not at risk for gastrointestinal (GI) disease may be as described herein.

In some embodiments, the step of identifying a subject dog as being at risk or not at risk for gastrointestinal (GI) disease in the method for preventing or treating GI disease comprises one or more of: (a) analyzing a fecal sample from the subject dog to determine fecal sample values for one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes. In some embodiments, the method also comprises (b) comparing the values obtained from (a) with control values from one or more not at risk for GI disease dog for a respective one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes. In some embodiments, the subject dog is determined to be not at risk for GI disease when one or more of the fecal sample values is similar or equal to the control values. In some embodiments, the subject dog is determined to be at risk of GI disease when on or more of the fecal sample values is different than the control values.

In some embodiments, the subject dog is determined to be not at risk for GI disease when each of the fecal sample values is similar or equal to the control values, or wherein the subject dog is determined to be at risk of GI disease when each of the fecal sample values is different than the control values.

In some embodiments, the subject dog is determined to be not at risk for GI disease when the fecal sample has, compared to the control values: (i) an approximately equal to or higher level total short chain fatty acid content; (ii) an approximately equal to or higher level of C2 SCFA; (iii) an approximately equal to or higher level of C3 SCFA; (iv) an approximately equal to or higher level of C4 SCFA; (v) an approximately equal to or lower pH; (vi) an approximately equal to or higher fecal score; (vii) an approximately equal to or higher abundance of Bacteriodetes bacteria, Prevotellaceae bacteria, Ruminococcaceae bacteria, Bacteroidaceae bacteria, and Prevotella bacteria, and an approximately equal to or lower abundance of Enterobacteriaceae bacteria. In some embodiments, the subject dog is determined to be at risk for GI disease when the feces sample has, compared to the control values: (i) a lower level of total short chain fatty acid content; (ii) a lower level of C2 SCFA; (iii) a lower level of C3 SCFA; (iv) a lower level of C4 SCFA; (v) a higher pH; (vi) a lower fecal score; (vii) a lower abundance of Bacteriodetes bacteria, Prevotellaceae bacteria, Ruminococcaceae bacteria, Bacteroidaceae bacteria, and Prevotella bacteria, and a higher abundance of Enterobacteriaceae bacteria.

In some embodiments, the subject dog is determined to be not at risk for GI disease when the feces sample has, compared to the control values, at least one of: (i) an approximately equal to or higher level total short chain fatty acid content; (ii) an approximately equal to or higher level of C2 SCFA; (iii) an approximately equal to or higher level of C3 SCFA; (iv) an approximately equal to or higher level of C4 SCFA; (v) an approximately equal to or lower pH; (vi) an approximately equal to or lower moisture, (vii) an approximately equal to or higher fecal score; and (viii) one or both an approximately equal to or higher abundance of bacteria of one or more of Prevotellaceae, Ruminococcaceae, and Bacteroidaceae, and an approximately equal to or lower abundance of bacteria of one or both of Enterobacteriaceae and Bacteriodetes other than Prevotellaceae and Bacteroidaceae. In some embodiments, the subject dog is determined to be at risk for GI disease when the feces sample has, compared to the control values, at least one of: (i) a lower level of total short chain fatty acid content; (ii) a lower level of C2 SCFA; (iii) a lower level of C3 SCFA; (iv) a lower level of C4 SCFA; (v) a higher pH; (vi) a higher moisture, (vii) a lower fecal score; and (viii) one or more of a lower abundance of bacteria of one or more of Prevotellaceae, Ruminococcaceae, and Bacteroidaceae, and a higher abundance of bacteria of one or both of Enterobacteriaceae and Bacteriodetes other than Prevotellaceae and Bacteroidaceae.

In some embodiments, the step of identifying a subject dog as being at risk or not at risk for gastrointestinal (GI) disease comprises: (a) analyzing a fecal sample from the subject dog to determine fecal sample values for one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (vii) abundance of bacteria of Prevotellaceae, Ruminococcaceae, Bacteroidaceae, Enterobacteriaceae, and Bacteroidetes.

In some embodiments, the subject dog is determined not to be at risk for GI disease when the fecal sample values include one or more of: (i) the total short chain fatty acid content (SCFA) as found for not at risk dogs in examples herein; (ii) the level of C2 SCFA as found for not at risk dogs in examples herein; (iii) the level of C3 SCFA as found for not at risk dogs in examples herein; (iv) the level of C4 SCFA as found for not at risk dogs in examples herein; (v) the pH as found for not at risk dogs in examples herein; (vi) the moisture as found for not at risk dogs in examples herein; (vii) the fecal score as found for not at risk dogs in examples herein; and (vii) an abundance of bacteria of one or more of Prevotellaceae, Ruminococcaceae, Bacteroidaceae, Enterobacteriaceae, and Bacteroidetes as found for not at risk dogs in examples herein. In some embodiments, the fecal sample values include each of (i) through (vii). In some embodiments, the fecal sample values include each of (i) through (vii) and the abundance bacteria of each of Prevotellaceae, Ruminococcaceae, Bacteroidaceae, Enterobacteriaceae, and Bacteroidetes.

In some embodiments, the subject dog is determined to be at risk for GI disease when the fecal sample values include one or more of: (i) the total short chain fatty acid content (SCFA) less than that observed for not at risk dogs in examples herein; (ii) the level of C2 SCFA less than that observed for not at risk dogs in examples herein; (iii) the level of C3 SCFA less than that observed for not at risk dogs in examples herein; (iv) the level of C4 SCFA less than that observed for not at risk dogs in examples herein; (v) the pH greater than that observed for not at risk dogs in examples herein; (vi) the moisture greater than that observed for not at risk dogs in examples herein; (vii) the fecal score less than that observed for not at risk dogs in examples herein; and (viii) one or more of an abundance of bacteria of one or more of Prevotellaceae, Ruminococcaceae, and Bacteroidaceae less than that observed for not at risk dogs in examples herein, and one or both of an abundance of bacteria of Enterobacteriaceae and Bacteriodetes, other than Prevotellaceae and Bacteroidaceae, greater than that observed for not at risk dogs in examples herein. In some embodiments, the fecal sample values include each of (i) through (vii). In some embodiments, the fecal sample values include each of (i) through (vii) and the abundance of bacteria of each of Prevotellaceae, Ruminococcaceae, Bacteroidaceae, Enterobacteriaceae, and Bacteroidetes.

In some embodiments, the GI disease-mitigating diet comprises a dog food composition. In some embodiments, the dog food composition comprises one or more of quinoa, amaranth, egg shell membrane, and dietary fiber. In some embodiments, the composition comprises each of quinoa, amaranth, egg shell membrane, and dietary fiber. In some embodiments, the composition comprises from about 1 to about 10 wt. % of quinoa; from about 1 to about 10 wt. % of amaranth; from about 0.5 to about 8 wt. % of egg shell membrane, and from about 2 to about 20 wt. % of dietary fiber. In some embodiments, the dietary fiber comprises insoluble fiber and soluble fiber. In some embodiments, the dietary fiber comprises i) from about 1 to about 18 wt. % of insoluble fiber, and ii) from about 0.5 to about 8 wt. % of soluble fiber. The weight percentages are based on the total weight of the dog food composition on a dry matter basis.

In some embodiments, the dog food composition further comprises from about 0.1 to about 10 wt. % of fatty acids. In some embodiments, the fatty acids comprise a polyunsaturated fatty acid. In some embodiments, the polyunsaturated fatty acid comprises an omega-3 fatty acid, or an omega-6 fatty acid, or a combination of two or more thereof. In some embodiments, the polyunsaturated fatty acid comprises an omega-3 fatty acid selected from linolenic acid, stearidonic acid, cicosatetraenoic acid, cicosapentaenoic acid, docosapentaenoic acid, or docosahexaenoic acid, or a combination of two or more thereof. In some embodiments, the omega-3 fatty acid comprises alpha-linolenic acid. In some embodiments, the omega-3 fatty acid comprises eicosapentaenoic acid or docosahexaenoic acid, or a combination thereof.

In some embodiments, the polyunsaturated fatty acid comprises an omega-6 fatty acid selected from linolenic acid, calendic acid, eicosadienoic acid, arachidonic acid, docosadienoic acid, adrenic acid, osbond acid, tetracosatetraenoic acid, or tetracosapentaenoic acid, or a combination of two or more thereof. In some embodiments, the polyunsaturated fatty acid comprises an omega-6 fatty acid selected from linolenic acid, arachidonic acid, and a combination of two or more thereof.

An embodiment comprises a device comprising an input, a memory, a processor, and an output. In some embodiments, the device is an electronic mobile device, a computer, or a cell phone. The input is configured to receive the results of a method for identifying a subject dog as being at risk or not at risk for gastrointestinal (GI) disease. The memory comprises processor executable instructions for comparing the results to control values, and for preparing a conclusion of at risk or not at risk based on the comparing, and outputting the results. The processor is configured to execute the processor executable instructions. The output is configured to display the conclusions. In some embodiments, the processor executable instructions further comprise instructions to display a recommendation to feed to the subject a GI disease-mitigating diet when conclusion is that the subject dog is at risk, the processor is further configured to execute the instructions to display a recommendation, and the output is further configured to display the recommendation. In some embodiments, the GI disease-mitigating diet is a dog food composition described herein. In some embodiments, the dog food composition comprises one or more of quinoa, amaranth, egg shell membrane, and dietary fiber. In some embodiments, the composition comprises each of quinoa, amaranth, egg shell membrane, and dietary fiber. In some embodiments, the composition comprises from about 1 to about 10 wt. % of quinoa; from about 1 to about 10 wt. % of amaranth; from about 0.5 to about 8 wt. % of egg shell membrane, and from about 2 to about 20 wt. % of dietary fiber. In some embodiments, the dietary fiber comprises insoluble fiber and soluble fiber. In some embodiments, the dietary fiber comprises i) from about 1 to about 18 wt. % of insoluble fiber, and ii) from about 0.5 to about 8 wt. % of soluble fiber. The weight percentages are based on the total weight of the dog food composition on a dry matter basis. In some embodiments, the method for identifying a subject dog as being at risk or not at risk for gastrointestinal (GI) disease generating the results is any one method herein.

Certain aspects of further embodiments herein follow. The aspects are further embodiments herein. The embodiments above are not necessarily limited to elements of any one or more of the aspects below. However, still further embodiments herein include providing any one or more element of the following aspects in one of the above embodiments, or substituting any one or more element of the following aspects for one or more elements in one of the above embodiments.

In accordance with a first aspect, provided is a method for identifying a subject dog as being at risk or not at risk for gastrointestinal (GI) disease comprising:
(a) analyzing a feces sample from the dog for pH, total short chain fatty acid content (SCFA), and three or more of: C2 SCFA, C3 SFCA, C4 SCFA, C5 SCFA, and/or C6 SCFA content, feces score, moisture, and relative abundance of Prevotellaceae, Phylum Bacteroidetes, excluding F3: f_Bacteroidaceae and F4: f_Prevotellaceae, Ruminococcaceae, Enterobacteriaceae, Clostridiales and/or Prevotella, and optionally other bacterial phyla, families, genera or species;
(b) comparing the values obtained from (a) with control values obtained from one dog or pooled from a plurality of dogs that have been medically determined to not be at risk for GI disease;
wherein the subject dog is identified as not being at risk for GI disease when the feces sample has:
(i) approximately equal or lower pH and approximately equal or higher levels of total SFCA than the control values, and
(ii) when measured, approximately equal or lower values for moisture and/or relative abundance of Enterobacteriaceae than the control values, and approximately equal or higher levels of C3 SCFA, C2 SFCA, C4 SCFA, feces score, and relative abundance of Prevotellaceae, Phylum Bacteroidetes, excluding F3: f_Bacteroidaceae and F4: f_Prevotellaceae, Ruminococcaceae, Clostridiales and/or Prevotella than the control values.

The presence of other bacteria may be determined because greater diversity in the gut microbiome is also generally associated with No Risk dogs.

For purposes of this aspect, the term “three or more of” specifically includes four or more of, five or more of, six or more of, seven or more of, eight or more of, nine or more of, ten or more of, eleven or more of, twelve or more of, thirteen or more of, or fourteen or more of.

In some embodiments of this aspect, the “relative abundance” of bacteria can be determined as described in Examples 3 and 4.

For purposes of this aspect, the term “approximately” means with a variance of 10% or less, except as where stated otherwise.

For purposes of this aspect, the term “control values obtained from one dog or pooled from a plurality of dogs that have been medically determined to not be at risk for GI disease” can be from a concurrent experiment, or more likely through archived values from previous experiments, including, without limitation, values from this specification.

For purposes of this aspect, the term “not at risk” generally means as determined by licensed veterinarians.

In accordance with a second aspect, provided is a kit for identifying a subject dog as being at risk or not at risk for GI disease comprising: a reagent for determining pH of a fecal sample, one or more reagent for measuring volatile carboxylic acids in a fecal sample, and one or more reagent for quantitatively identifying in a fecal sample one or more of Prevotellaceae, Phylum Bacteroidetes-excluding F3: f_Bacteroidaceae and F4: f_Prevotellaccac, Ruminococcaceae, Enterobacteriaceae, Clostridiales, Prevotella and optionally other bacteria.

For purposes of this aspect, the term “reagent for determining pH” includes, without limitation, pH meters, pH test sticks, liquid pH indicators, metal-electrode devices, glass electrode devices, semiconductor sensors, hydrogen-electrode devices, quinhydrone-electrode devices, ISFET (ion-sensitive field-electrode transistor) electrodes and antimony-electrode devices.

For purposes of this aspect, the term “one or more reagent for measuring volatile carboxylic acids” include, without limitation, those taught in Example 2 hereof, bromocresol green reagent, gas chromatography/mass spectrometry and high-performance liquid chromatography/mass spectrometry devices.

For purposes of this aspect, “one or more reagents for quantitatively identifying in a fecal sample one or more of Prevotellaceae, Phylum Bacteroidetes, excluding F3: f_Bacteroidaceae and F4: f_Prevotellaceac, Ruminococcaceae, Enterobacteriaceae, Clostridiales and Prevotella, and optionally other bacteria” include, without limitation, commonly used polymerase chain reaction (PCR) reagents with 16S amplicon sequencing technology, shotgun metagenomics technology, which includes any available sequencing platforms (such as Illumina, 454 sequencing, nanopore and PacBio), multiplex qPCR method(s) based on designing specific primers for individual components at phyla, family, genus and species level and methods using aptamers.

In accordance with another aspect of the invention, provided is a method for preventing or treating GI disease comprising identifying a GI status of a dog; i.e., identifying whether the dog is at risk or not at risk for gastrointestinal (GI) disease, conducting the method according to the first aspect of the invention with a sample collected from the dog, and providing to the dog if it is determined to be at risk for GI disease with a GI disease-mitigating diet. as now described. Generally, dog foods are GI disease-mitigating diets tend to be high in total dietary fiber (at least 2% to about 20% by weight), have a useful soluble fiber to insoluble fiber ratio (e.g., from about 1:2 to about 1:18, and low in fat (e.g., 10% or less by weight, or 20% or less by total calories) and contain polyunsaturated fats, omega-3 fatty acids and omega-6 fatty acids.

In some aspects, the GI disease-mitigating diet is a dog food composition comprising: a controlled release pet food composition comprising: a matrix comprising: a fiber component comprising a high solubility fiber source and low solubility fiber source, and a polyphenol source; wherein the matrix is adapted to deliver the polyphenol source to the lower gastrointestinal (GI) tract of a mammal after ingestion by the mammal.

In some aspects, the GI disease-mitigating diet comprises a weight ratio of high solubility fiber source to low solubility fiber source of from about 1:20 to about 1:1. In some aspects, the weight ratio of high solubility fiber source to low solubility fiber source is from about 1:15 to about 1:2. In other aspects, the weight ratio of high solubility fiber source to low solubility fiber source is from about 1:10 to about 1:3. In further aspects, the weight ratio of high solubility fiber source to low solubility fiber source is from about 1:5 to about 1:3. In yet other aspects, the weight ratio of high solubility fiber source to low solubility fiber source is about 1:4.

In some aspects, the high solubility fiber source comprises oat bran, buckwheat groats, pea bran, barley, tomato pomace, citrus pulp, beet pulp, and a combination of two or more thereof. In some aspects, the low solubility fiber source comprises a cellulosic material, a pecan fiber, or a combination thereof.

In some aspects, the polyphenol source is of food or plant origin. In some aspects, the polyphenol source of food origin comprises a polyphenol of fruit or vegetable origin.

In some aspects, the polyphenol source comprises a flavonoid or a phenolic acid. In certain aspects, the polyphenol source provides a polyphenol selected from: dehydroxy rosmarinic acid, Coumaroylnepitrin, cupafolin, carnosol, scutellarin, kaempferol, rosmarinic acid, rosmanol, cirsimaritin, luteolin, 6-methoxy-luteolin, 7-epirosmannol, quercetin, catechin, hesperidin, cyanidin, and a combination of two or more thereof.

In some aspects, the GI-disease mitigating diet further comprises a source of hydrolyzed animal or plant protein comprising an amino acid profile. In some aspects, the source of hydrolyzed animal or plant protein comprises chicken liver. In other aspects, the source of hydrolyzed animal or plant protein is present in an active content of from about 25 to about 45 wt. %.

In some aspects, the GI-disease mitigating diet further comprises high docosahexaenoate fish oil. In other aspects, the active content of the high docosahexaenoate fish oil is from about 0.5 to about 2.5 wt. %.

In some aspects, the fiber component comprises pecan shells; flax seed; citrus pulp; beet pulp; cranberry pomace; or a combination of two or more thereof. In some aspects, the GI-disease mitigating diet comprises from about 1 to about 10 wt. % of pecan shells; from about 1 to about 5 wt. % of flax seed; from about 1 to about 5 wt. % of citrus pulp; from about 1 to about 5 wt. % of bect pulp; and from about 0.1 to about 2 wt. % of cranberry pomace. In other aspects, the GI-disease mitigating diet comprises from about 2.5 to about 8 wt. % of pecan shells; from about 2 to about 4 wt. % of flax seed; from about 2 to about 3 wt. % of citrus pulp; from about 2 to about 3 wt. % of beet pulp; and from about 0.5 to about 1.5 wt. % of cranberry pomace. In certain aspects, the GI-disease mitigating diet comprises about 7 wt. % of pecan shells; about 3 wt. % of flax seed; about 2.5 wt. % of citrus pulp; about 2.5 wt. % of beet pulp; and about 1 wt. % of cranberry pomace. Still further aspects provide a GI-disease mitigating diet comprising about 3.5 wt. % of pecan shells; about 1.5 wt. % of flax seed; about 1.25 wt. % of citrus pulp; about 1.25 wt. % of beet pulp; and about 0.5 wt. % of cranberry pomace.

According to other aspects, the pet food composition of further comprises from about 0.1 to about 10 wt. % of fatty acids.

According to a third aspect, the dog food composition of the first aspect, wherein the fatty acid comprises a polyunsaturated fatty acid.

According to a fourth aspect, the dog food composition of the third aspect, wherein the polyunsaturated fatty acid comprises an omega-3 fatty acid, an omega-6 fatty acid, or a combination of two or more thereof.

According to a fifth aspect, the dog food composition of the fourth aspect, wherein polyunsaturated fatty acid comprises an omega-3 fatty acid selected from linolenic acid, stearidonic acid, cicosatetraenoic acid, eicosapentaenoic acid, docosapentaenoic acid, docosahexaenoic acid, and a combination of two or more thereof.

According to a sixth aspect, the dog food composition of the fourth or fifth aspect, wherein the omega-3 fatty acid comprises alpha-linolenic acid.

According to a seventh aspect, the dog food composition according to one of the fourth to the sixth aspects, wherein the omega-3 fatty acid comprises eicosapentaenoic acid, docosahexaenoic acid, or a combination thereof.

According to an eighth aspect, the dog food composition according to one of the fourth to seventh aspects, wherein the polyunsaturated fatty acid comprises an omega-6 fatty acid selected from linolenic acid, calendic acid, eicosadienoic acid, arachidonic acid, docosadienoic acid, adrenic acid, osbond acid, tetracosatetraenoic acid, tetracosapentaenoic acid, and a combination of two or more thereof.

According to a ninth aspect, the dog food composition according to the fourth or the eighth aspects, wherein the polyunsaturated fatty acid comprises an omega-6 fatty acid selected from linolenic acid, arachidonic acid, and a combination of two or more thereof.

According to a tenth aspect, the dog food composition according to the eighth or the ninth aspects, wherein the dog food composition has a weight ratio of linolenic acid to arachidonic acid of from about 12:1 to about 50:1, preferably about 15:1 to about 40:1, preferably about 18:1 to about 35:1, preferably about 20:1 to about 30:1, preferably about 22:1 to about 28:1, or preferably about 26:1 to about 28:1.

According to an eleventh aspect, the dog food composition according to one of the fourth to the tenth aspects, wherein the food composition has a weight ratio of omega-3 fatty acids to omega-6 fatty acids of from about 0.5:1 to about 7:1, preferably, about 0.5:1 to about 3:1, preferably about 0.5:1 to about 2.5:1, preferably about 0.5:1 to about 2:1, preferably about 0.5:1 to about 1.5:1, preferably about 0.5:1 to about 1:1; about 0.6:1 to about 7:1, preferably, about 0.6:1 to about 3:1, preferably about 0.6:1 to about 2.5:1, preferably about 0.6:1 to about 2:1, preferably about 0.6:1 to about 1.5:1, or preferably about 0.6:1 to about 1:1.

According to a twelfth aspect, the dog food composition according to any preceding aspect, wherein the dog food composition has a macronutrient value comprising:
10 wt. % of fat or less;
from about 10 to about 40 wt. % of protein; and
from about 2 to about 20 wt. % of dietary fiber, the dietary fiber comprising:
   i) from about 1 to about 18 wt. % of insoluble fiber, and
   ii) from about 0.5 to about 8 wt. % of soluble fiber.

According to a thirteenth aspect, the dog food composition according to the twelfth aspect, wherein the food composition has a weight ratio of soluble fiber to insoluble fiber of from about 1:20 to about 10:1, or about 1:18 to about 5:1, or about 1:18 to about 2:1, or about 1:18 to about 1:1, or about 1:18 to about 1:2.

According to a fourteenth aspect, the dog food composition according to any foregoing aspect, wherein the dog food composition comprises a core and a coating disposed on the core, the core comprising the lysine, the quinoa, and the amaranth, and the coating comprising the egg shell membrane.

In accordance with a fifteenth aspect, a dog food composition is provided comprising:
an alpha-amino acid;
one or more ancient grain;
egg shell membrane;
wherein the dog food composition has a macronutrient value comprising:
   from about 1 to about 8 wt. % of omega-3 fatty acid; and
   from about 1 to about 15 wt. % of omega-6 fatty acid,
   wherein the food composition has a weight ratio of omega-3 fatty acids to omega-6 fatty acids of from about 0.5:1 to about 7:1, and all weight percentages are based on the total weight of the dog food composition on a dry matter basis.

According to a sixteenth aspect, the dog food composition according to the fifteenth aspect, wherein the alpha-amino acid contains an alpha-carboxylic acid group.

According to a seventeenth aspect, the dog food composition according to the fifteenth or sixteenth aspects, wherein the alpha-amino acid comprises lysine.

According to an eighteenth aspect, the dog food composition according to one of the fifteenth to seventeenth aspects, wherein the one or more ancient grain is selected from spelt, Khorasan wheat, einkorn, emmer, millet, barley, teff, sorghum, quinoa, amaranth, buckwheat, chia, and a combination of two or more thereof.

According to a nineteenth aspect, the dog food composition according to the eighteenth aspect, wherein the one or more ancient grain comprises an amaranth.

According to a twentieth aspect, the dog food composition according to one of the fifteenth to nineteenth aspects, wherein dog food composition comprises at least two ancient grains.

According to a twenty first aspect, the dog food composition according to one of the fifteenth to the twentieth aspects, wherein the weight ratio of omega-3 fatty acids to omega-6 fatty acids is from about 0.5:1 to about 3:1, preferably about 0.5:1 to about 2.5:1, preferably about 0.5:1 to about 2:1, preferably about 0.5:1 to about 1.5:1, or preferably about 0.5:1 to about 1:1.

According to a twenty second aspect, the dog food composition according to one of the fifteenth to the twenty first aspects, wherein the omega-3 fatty acid selected from linolenic acid, stearidonic acid, eicosatetraenoic acid, eicosapentaenoic acid, docosapentaenoic acid, docosahexaenoic acid, and a combination of two or more thereof.

According to a twenty third aspect, the dog food composition according to one of the fifteenth to the twenty second aspects, wherein the omega-6 fatty acid selected from linolenic acid, calendic acid, eicosadienoic acid, arachidonic acid, docosadienoic acid, adrenic acid, osbond acid, tetracosatetraenoic acid, tetracosapentaenoic acid, and a combination of two or more thereof. In accordance with a twenty fourth aspect, provided is a dog food composition comprising:
(a) a core, the core comprising:
   quinoa;
   amaranth; and
(b) a coating disposed on the core, the coating comprising:
   egg shell membrane,
   wherein the dog food composition has a macronutrient value comprising from about 2 to about 10 wt. % of dietary fiber, the dietary fiber comprising:
   from about 1 to about 9 wt. % of insoluble fiber, and
   from about 0.2 to about 4 wt. % of soluble fiber,
   wherein the food composition has a weight ratio of soluble fiber to insoluble fiber of from about 1:20 to about 2:1, and all weight percentages are based on the total weight of the dog food composition on a dry matter basis.

According to a twenty sixth aspect, the dog food composition according to one of the twenty fourth or the twenty fifth aspects, wherein the macronutrient value of the dog food composition further comprising from about 0.1 to about 10 wt. % of a fatty acid.

According to a twenty seventh aspect, the dog food composition according to the twenty sixth aspect, wherein the fatty acid comprises a polyunsaturated fatty acid.

According to a twenty eighth aspect, the dog food composition according to the twenty seventh aspect, wherein the polyunsaturated fatty acid comprises an omega-3 fatty acid, an omega-6 fatty acid, or a combination of two or more thereof.

According to a twenty ninth aspect, the dog food composition according to the twenty eighth aspect, wherein polyunsaturated fatty acid comprises an omega-3 fatty acid selected from linolenic acid, stearidonic acid, eicosatetraenoic acid, eicosapentaenoic acid, docosapentaenoic acid, docosahexaenoic acid, and a combination of two or more thereof.

According to a thirtieth aspect, the dog food composition according to the twenty eighth or twenty ninth aspects, wherein the omega-3 fatty acid comprises alpha-linolenic acid.

According to a thirty first aspect, the dog food composition according to one of the twenty eighth to thirtieth aspects, wherein the omega-3 fatty acid comprises eicosapentaenoic acid, docosahexaenoic acid, or a combination thereof.

According to a thirty second aspect, the dog food composition according to one of the twenty eighth to thirtieth aspects, the dog food composition of one of the twenty fourth to the twenty sixth aspects, wherein the polyunsaturated fatty acid comprises an omega-6 fatty acid selected from linolenic acid, calendic acid, cicosadienoic acid, arachidonic acid, docosadienoic acid, adrenic acid, osbond acid, tetracosatetraenoic acid, tetracosapentaenoic acid, and a combination of two or more thereof.

According to a thirty third aspect, the dog food composition according to the twenty eighth or thirty second aspects, wherein the polyunsaturated fatty acid comprises an omega-6 fatty acid selected from linolenic acid, arachidonic acid, and a combination of two or more thereof.

According to a thirty fourth aspect, the dog food composition according to the thirty second or the thirty third aspects, wherein the dog food composition has a weight ratio of linolenic acid to arachidonic acid of from about 12:1 to about 50:1, preferably about 15:1 to about 40:1, preferably about 18:1 to about 35:1, preferably about 20:1 to about 30:1, or preferably about 22:1 to about 28:1.

According to a thirty fifth aspect, the dog food composition according to one of the twenty eighth to thirty fourth aspects, wherein the food composition has a weight ratio of omega-3 fatty acids to omega-6 fatty acids of from about 0.5:1 to about 7:1, preferably, about 0.5:1 to about 3:1, preferably about 0.5:1 to about 2.5:1, preferably about 0.5:1 to about 2:1, preferably about 0.5:1 to about 1.5:1, preferably about 0.5:1 to about 1:1, about 0.6:1 to about 7:1, preferably, about 0.6:1 to about 3:1, preferably about 0.6:1 to about 2.5:1, preferably about 0.6:1 to about 2:1, preferably about 0.6:1 to about 1.5:1, or preferably about 0.6:1 to about 1:1.

According to a thirty sixth aspect, the dog food composition according to one of the twenty eighth to the thirty fifth aspects, wherein the food composition further comprising an alpha-amino acid.

According to a thirty seventh aspect, the dog food composition according to the thirty sixth aspect, wherein the alpha-amino acid contains an alpha-carboxylic acid group.

According to a thirty eighth aspect, the dog food composition according to the thirty seventh aspect, wherein the alpha-amino acid comprises lysine.

According to a thirty ninth aspect, the dog food composition according to any foregoing aspect, wherein the dog food composition is in the form of a dog treat and/or a supplement adapted for combination with dog food.

In accordance with a fortieth aspect, provided is a dog food composition comprising:
an amount of quinoa;
an amount of amaranth; and
an amount of egg shell membrane,
from about 2 to about 10 wt. % of dietary fiber, the dietary fiber comprising:
from about 1 to about 9 wt. % of insoluble fiber, and
from about 0.2 to about 4 wt. % of soluble fiber,
   wherein the dog food composition has a weight ratio of quinoa to amaranth of from about 1:12 to about 12:1, optionally from about 1:10 to about 10:1, optionally from about 1:8 to about 8:1, optionally from about 1:7 to about 7:1, optionally from about 1:6 to about 6:1, optionally from about 1:5 to about 5:1, optionally from about 1:4 to about 4:1, optionally from about 1:3 to about 3:1, or optionally from about 1:2 to about 2:1;
   the dog food composition having a weight ratio of quinoa to egg shell membrane of from about 1:10 to about 12:1, optionally from about 1:8 to about 10:1, optionally from about 1:6 to about 8:1, optionally from about 1:5 to about 7:1, optionally from about 1:4 to about 6:1, optionally from about 1:3 to about 5:1, optionally from about 1:2 to about 4:1, optionally from about 1:1 to about 3:1, or optionally from about 1:1 to about 2:1; and
   the dog food composition having a weight ratio of amaranth to egg shell membrane of from about 1:10 to about 12:1, optionally from about 1:8 to about 10:1, optionally from about 1:6 to about 8:1, optionally from about 1:5 to about 7:1, optionally from about 1:4 to about 6:1, optionally from about 1:3 to about 5:1, optionally from about 1:2 to about 4:1, optionally from about 1:1 to about 3:1, or optionally from about 1:1 to about 2:1.

According to a forty first aspect, the dog food composition according to the fortieth aspect, wherein the dog food composition has a weight ratio of quinoa to amaranth to egg shell membrane (quinoa: amaranth: egg shell membrane) of from about 1:10:8 to about 10:10:1.

According to a forty second aspect, the dog food composition according to the forty fortieth or forty fifth aspect, comprising:
from about 1 to about 10 wt. % of quinoa;
from about 1 to about 10 wt. % of amaranth; and
from about 0.5 to about 8 wt. % of egg shell membrane,
from about 2 to about 10 wt. % of dietary fiber, the dietary fiber comprising:
from about 1 to about 9 wt. % of insoluble fiber, and
from about 0.2 to about 4 wt. % of soluble fiber,
   wherein all weight percentages are based on the total weight of the dog food composition on a dry matter basis.

The following characteristics may apply to any or all aspects of this aspect of the invention.

To the extent that food and food ingredient contain water/moisture, the dry matter represents everything in the sample other than water including, for example, protein, fiber, fat, carbohydrates, minerals, etc. Dry matter weight is the total weight minus the weight of any water. The skilled artisan would readily recognize and understand nutritional amounts and percentages expressed as dry matter amounts, dry matter weights and dry matter percentages.

Dry matter intake per day is calculated as the total nutritional intake per day excluding all water. For example, an amount of an ingredient equal to a specific percent of daily nutritional intake refers to the amount of that ingredient in dry matter form (i.e., excluding all water) relative to the total amount of dry matter consumed (also excluding all water) in a day.

“Daily nutritional intake” and “total nutritional intake per day” refer to dry matter intake per day. That is, water weight is not included in calculating the amount of nutrition consumed per day. To calculate percent of an ingredient of total daily intake on a dry matter basis, water is removed from the total intake to give total daily dry matter intake and the percent of the ingredient is based on amount of ingredient present as dry matter.

As used herein, an “ingredient” refers to any component of a dog food composition. The term “nutrient” refers to a substance that provides nourishment and thus has a nutrient value. In some cases, an ingredient may comprise more than one “nutrient,” for example, a composition may comprise corn comprising important nutrients including both protein and carbohydrate.

The dog food compositions according to certain aspects of the invention include from about 0.1 to about 5 wt. % of an alpha-amino acid; from about 1 to about 15 wt. % of one or more ancient grains (e.g., about 1 to about 10 wt. % of quinoa and about 1 to about 10 wt. % of amaranth); and from about 0.5 to about 8 wt. % of egg shell membrane, wherein all weight percentages are based on the total weight of the dog food composition on a dry matter basis. The dog food compositions may, in some aspects, include one or more fatty acid, such as a polyunsaturated fatty acid. For example, the dog food composition may include a polyunsaturated fatty acid(s) selected from omega-3 fatty acid, an omega-6 fatty acid, or a combination of two or more thereof. The dog food compositions may be formulated to have omega-3 fatty acid(s) and an omega-6 fatty acid(s), preferably in a weight ratio of from about 0.5:1 to about 7:1. In some aspects, the dog food compositions may be formulated to control the weight ratio of linolenic acid to arachidonic acid of from about 12:1 to about 50:1.

The dog food composition may also have a macronutrient value comprising from about 2 to about 20 wt. % of dietary fiber, where the dietary fiber comprises from about 1 to 18 wt. % of insoluble fiber, and from about 0.5 to 4 wt. % of soluble fiber. Additionally, the dog food composition may have a weight ratio of soluble fiber to insoluble fiber of from about 1:20 to about 5:1, where all weight percentages are based on the total weight of the dog food composition on a dry matter basis.

In accordance with further aspects, the dog food composition is a dog food composition is in a form having a core and a coating disposed on the core. The core typically comprises lysine and amaranth, while the coating comprising egg shell membrane. Without being limited to any particular theory, it is believed that including the eggshell membrane in the coating may maintain higher concentrations of the bioactive peptides in the egg shell membrane.

Suitable components, such as those listed below, may be included or excluded from the formulations for the dog food compositions depending on the specific combination of other ingredients and the form of the dog food compositions. In some aspects, the dog food compositions disclosed herein may be in the form of a stand-alone dog food, as a supplement to dog food, as a dog food treat, or the like.

The dog food compositions typically include one or more ancient grain. As used herein, an ancient grain refers to wheats, such as spelt, Khorasan wheat (Kamut), cinkorn, and emmer; grains, such as millet, barley, teff, oats, and sorghum; and pseudocereals such as, amaranth, quinoa, buckwheat, and chia. In some aspects, the dog food composition comprises two or more ancient grains, three or more ancient grains, four or more ancient grains, or five or more ancient grains. The ancient grains may be selected in some cases from millet, barley, teff, oats, sorghum, amaranth, buckwheat, chia, and a combination of two or more thereof. Preferably, the ancient grains are selected from quinoa, amaranth, buckwheat, bulgar, sorghum, and a combination of two or more thereof. In some cases, the dog food composition may include at least two ancient grains selected from quinoa, amaranth, buckwheat, bulgar, sorghum, and a combination of two or more thereof. For example, the dog food composition may comprise quinoa and an amaranth.

Additionally or alternatively, the dog food composition may comprise a first ancient grain (e.g., quinoa) and a second ancient grain (e.g., amaranth) in a weight ratio of from about 1:12 to about 12:1. In some instances, the dog food composition has a weight ratio of a first ancient grain to second ancient grain of from about 1:10 to about 10:1, from about 1:8 to about 8:1, from about 1:7 to about 7:1, from about 1:6 to about 6:1, from about 1:5 to about 5:1, from about 1:4 to about 4:1, from about 1:3 to about 3:1, or from about 1:2 to about 2:1, or any range or subrange thereof. In at least one aspect, the weight ratio of the first ancient grain (e.g., quinoa) to the second ancient grain (e.g., amaranth) is about 1:1 or is 1:1.

The one or more ancient grain may be included in the dog food compositions in an amount ranging from about 1 to about 15 wt. %, based on the total weight of the dog food composition on a dry matter basis. For example, the one or more ancient grain may be present in an amount of from about 1 to about 15 wt. %, about 1 to about 12 wt. %, about 1 to about 10 wt. %, about 1 to about 8 wt. %, about 1 to about 6 wt. %, about 1 to about 4 wt. %, about 1 to about 3 wt. %; from about 2 to about 15 wt. %, about 2 to about 12 wt. %, about 2 to about 10 wt. %, about 2 to about 8 wt. %, about 2 to about 6 wt. %, about 2 to about 4 wt. %, about 2 to about 3 wt. %; from about 3 to about 15 wt. %, about 3 to about 12 wt. %, about 3 to about 10 wt. %, about 3 to about 8 wt. %, about 3 to about 6 wt. %, about 3 to about 4 wt. %; from about 4 to about 15 wt. %, about 4 to about 12 wt. %, about 4 to about 10 wt. %, about 4 to about 8 wt. %, or about 4 to about 6 wt. %, including ranges and subranges thereof, based on the total weight of the dog food composition on a dry matter basis. In at least one aspect, the dog food composition includes about 2.5 wt. % or 2.5 wt. % of a first ancient grain, such as quinoa, based on the total weight of the dog food composition on a dry matter basis. Additionally or alternatively, the dog food composition may include about 2.5 wt. % or 2.5 wt. % of a second ancient grain, such as an amaranth, based on the total weight of the dog food composition on a dry matter basis.

The dog food composition typically includes egg shell membrane. Egg shell membrane typically includes a protein component, hyaluronic acid, and certain bioactive peptides that may be beneficial in the dog food compositions disclosed herein. Without being limited to any particular theory, it is believed that dog food compositions containing egg shell membrane instead of egg or dried egg, significantly reduces allergic reactions of dogs suffering from egg allergies. The egg shell membrane may be present in the dog food composition in an amount ranging from about 0.5 to about 8 wt. %, based on the total weight of the dog food composition on a dry matter basis. For instance, the dog food composition may include an amount of egg shell membrane from about 0.5 to about 8 wt. %, about 0.5 to about 7 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 8 wt. %, about 1 to about 7 wt. %, about 1 to about 6 wt. %, about 1 to about 5 wt. %, about 1 to about 4 wt. %, about 1 to about 3 wt. %, about 1 to about 2 wt. %; from about 2 to about 8 wt. %, about 2 to about 7 wt. %, about 2 to about 6 wt. %, about 2 to about 5 wt. %, about 2 to about 4 wt. %, about 2 to about 3 wt. %; from about 3 to about 8 wt. %, about 3 to about 7 wt. %, about 3 to about 6 wt. %, about 3 to about 5 wt. %, or about 3 to about 4 wt. %, including ranges and subranges thereof, based on the total weight of the dog food composition on a dry matter basis. In at least one aspect, the dog food composition includes about 2 wt. % or 2 wt. % of egg shell membrane, based on the total weight of the dog food composition on a dry matter basis.

In some aspects, the dog food composition is formulated to have certain weight ratios of the ancient grain(s) to the egg shell membrane. For example, the dog food composition may have a weight ratio of ancient grain(s) to egg shell membrane of from about 1:10 to about 24:1, from about 1:8 to about 20:1, from about 1:6 to about 16:1, from about 1:5 to about 14:1, from about 1:4 to about 12:1, from about 1:3 to about 10:1, from about 1:2 to about 8:1, from about 1:1 to about 6:1, from about 1:1 to about 4:1 or any ranges or subranges thereof. In at least one aspect, the dog food composition has at least two ancient grains with a weight ratio of a first ancient grain (e.g., quinoa) to egg shell membrane of from about 1:10 to about 12:1, from about 1:8 to about 10:1, from about 1:6 to about 8:1, from about 1:5 to about 7:1, from about 1:4 to about 6:1, from about 1:3 to about 5:1, from about 1:2 to about 4:1, from about 1:1 to about 6:1, from about 1:1 to about 5:1, from about 1:1 to about 4:1, from about 1:1 to about 3:1, from about 1:1 to about 2:1, or any ranges or subranges thereof. Additionally or alternatively, the dog food composition may have a weight ratio of a second ancient grain (e.g., amaranth) to egg shell membrane of from about 1:10 to about 12:1, from about 1:8 to about 10:1, from about 1:6 to about 8:1, from about 1:5 to about 7:1, from about 1:4 to about 6:1, from about 1:3 to about 5:1, from about 1:2 to about 4:1, from about 1:1 to about 6:1, from about 1:1 to about 5:1, from about 1:1 to about 4:1, from about 1:1 to about 3:1, from about 1:1 to about 2:1, or any ranges or subranges thereof.

The dog food composition, in some cases, preferably is formulated to have certain weight ratios of quinoa to amaranth to egg shell membrane (quinoa: amaranth: egg shell membrane) of from about 1:10:8 to about 10:10:1. For example, the weight ratio of quinoa to amaranth to egg shell membrane may be from about 1:10:8 to about 10:10:1, about 1:5:4 to about 5:5:1, about 1:8:4 to about 10:2:4, about 1:4:2 to about 5:1:2, or any ranges or subranges thereof.

Optionally, the dog food composition may include one or more an alpha-amino acid. The alpha-amino acid may contain an alpha-carboxylic acid group. In some aspects, the one or more alpha-amino acid comprises lysine. The alpha-amino acid may be present in the food composition in an amount ranging from about 0.1 to about 5 wt. %, based on the total weight of the dog food composition on a dry matter basis. For example, dog food composition may include one or more alpha-amino acid in an amount of from about 0.1 to about 5 wt. %, about 0.1 to about 4 wt. %, about 0.1 to about 3 wt. %, about 0.1 to about 2 wt. %, about 0.1 to about 1 wt. %; from about 0.2 to about 5 wt. %, about 0.2 to about 4 wt. %, about 0.2 to about 3 wt. %, about 0.2 to about 2 wt. %, about 0.2 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 0.75 to about 5 wt. %, about 0.75 to about 4 wt. %, about 0.75 to about 3 wt. %, about 0.75 to about 2 wt. %, about 0.75 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the dog food composition on a dry matter basis. In at least one aspect, the dog food composition includes an alpha-amino acid, preferably lysine, in an amount of about 2.5 wt. % or 2.5 wt. %, based on the total weight of the dog food composition on a dry matter basis.

Additional ingredients may be included, or in some instances excluded, include beef broth, brewers dried yeast, egg, egg product, flax meal, DL methionine, amino acids, leucine, lysine, arginine, cysteine, cystine, aspartic acid, polyphosphates, sodium pyrophosphate, sodium tripolyphosphate; zinc chloride, copper gluconate, stannous chloride, stannous fluoride, sodium fluoride, triclosan, glucosamine hydrochloride, chondroitin sulfate, green lipped mussel, blue lipped mussel, methyl sulfonyl methane (MSM), boron, boric acid, phytoestrogens, phytoandrogens, genistein, diadzein, Lcarnitine, chromium picolinate, chromium tripicolinate, chromium nicotinate, acid/base modifiers, potassium citrate, potassium chloride, calcium carbonate, calcium chloride, sodium bisulfate; eucalyptus, lavender, peppermint, plasticizers, colorants, flavorants, sweeteners, buffering agents, slip aids, carriers, pH adjusting agents, natural ingredients, stabilizers, biological additives such as enzymes (including proteases and lipases), chemical additives, coolants, chelants, denaturants, drug astringents, emulsifiers, external analgesics, fragrance compounds, humectants, opacifying agents (such as zinc oxide and titanium dioxide), antifoaming agents (such as silicone), preservatives (such as butylated hydroxytoluene (BHT) and butylated hydroxyanisole (BHA), propyl gallate, benzalkonium chloride, EDTA, benzyl alcohol, potassium sorbate, parabens and mixtures thereof), reducing agents, solvents, hydrotropes, solubilizing agents, suspending agents (non-surfactant), solvents, viscosity increasing agents (aqueous and non-aqueous), sequestrants, and/or keratolytics.

The dog food compositions are typically formulated so as to have one or more fatty acid(s). Non-limiting examples of fatty acids include, but are not limited to, omega-3 fatty acids, omega-6 fatty acids, lauric acid, myristic acid, palmitic acid, palmitoleic acid, margaric acid, margaroleic acid, stearic acid, oleic acid, stearidonic acid, gadoleic acid, behenic acid, crucic acid, docosatetra acid, and a combination of two or more thereof. The fatty acid(s) may be a polyunsaturated fatty acid, such as an omega-3 fatty acid, an omega-6 fatty acid, or a combination of two or more thereof. Non-limiting examples of omega-3 fatty acids include those selected from linolenic acid, stearidonic acid, eicosatetraenoic acid, eicosapentaenoic acid, docosapentaenoic acid, docosahexaenoic acid, and a combination of two or more thereof. The dog food composition may include linolenic acid, cicosapentaenoic acid, docosahexaenoic acid, or a combination of two or more thereof. In at least one aspect, the dog food composition comprises alpha-linolenic acid and/or gamma-linolenic acid. In further aspects, the dog food composition includes one or more omega-3 fatty acid(s) comprising eicosapentaenoic acid, docosahexaenoic acid, or a combination thereof.

Additionally or alternatively, the polyunsaturated fatty acid may comprise an omega-6 fatty acids. Examples of omega-6 fatty acid include linolenic acid, calendic acid, cicosadienoic acid, arachidonic acid, docosadienoic acid, adrenic acid, osbond acid, tetracosatetraenoic acid, tetracosapentaenoic acid, or a combination of two or more thereof. In some aspects, the polyunsaturated fatty acid comprises an omega-6 fatty acid selected from linolenic acid, arachidonic acid, and a combination of two or more thereof. In at least one preferable aspect, the dog food composition is formulated to have a weight ratio of linolenic acid to arachidonic acid of from about 12:1 to about 50:1. In some instances, the weight ratio of linolenic acid to arachidonic acid is about 15:1 to about 40:1, about 15:1 to about 35:1, about 15:1 to about 30:1, about 15:1 to about 28:1; from about 18:1 to about 40:1, about 18:1 to about 35:1, about 18:1 to about 30:1, about 18:1 to about 28:1; from about 20:1 to about 40:1, about 20:1 to about 35:1, about 20:1 to about 30:1, about 20:1 to about 28:1; from about 22:1 to about 40:1, about 22:1 to about 35:1, about 22:1 to about 30:1, about 2:1 to about 28:1; from about 18:1 to about 35:1, about 20:1 to about 30:1, about 22:1 to about 28:1, or any range or subrange thereof.

Additionally or alternatively, the dog food composition may be formulated to have a weight ratio of omega-3 fatty acids to omega-6 fatty acids of from about 0.5:1 to about 7:1. In some aspects, the dog food composition has a weight ratio of omega-3 fatty acids to omega-6 fatty acids of from about 0.5:1 to about 6:1, about 0.5:1 to about 5:1, about 0.5:1 to about 4:1, about 0.5:1 to about 3:1, about 0.5:1 to about 2.5:1, about 0.5:1 to about 2:1, about 0.5:1 to about 1.5:1, or about 0.5:1 to about 1:1; from about 1:1 to about 6:1, about 1:1 to about 5:1, about 1:1 to about 4:1, about 1:1 to about 3:1, about 1:1 to about 2.5:1, about 1:1 to about 2:1, about 1:1 to about 1.5:1, or about 1:1 to about 1:1, including ranges or subranges thereof.

In some aspects, the food composition further comprises one or more amino acids. The one or more amino acid(s) may be included in the dog food compositions as free amino acids, or supplied by any number of sources, e.g., crude protein, to the compositions of the present disclosure. Examples of amino acids may include, but are not limited to, Tryptophan, Taurine, Histidine, Carnitine, Carnosine, Alanine, Cysteine, Arginine, Methionine (including DL-methionine, and L-methionine), Tryptophan, Lysine, Asparagine, Aspartate (Aspartic acid), Phenylalanine, Valine, Threonine, Isoleucine, Histidine, Leucine, Glycine, Glutamine, Taurine, Tyrosine, Homocysteine, Ornithine, Citruline, Glutamate (Glutamic acid), Proline, and/or Serine.

The one or more amino acid(s) may comprise essential amino acids. Essential amino acids are amino acids that cannot be synthesized de novo, or in sufficient quantities by an organism and thus must be supplied in the diet. Essential amino acids vary from species to species, depending upon the organism's metabolism. For example, it is generally understood that the essential amino acids for dogs are phenylalanine, leucine, methionine, lysine, isoleucine, valine, threonine, tryptophan, histidine and arginine.

The dog food composition comprises a total dietary fiber typically in amount ranging from about 2 to about 20 wt. %, based on the total weight of the dog food composition on a dry matter basis. For example, the total dietary fiber may be present in an amount of from about 2 to about 20 wt. %, about 2 to about 16 wt. %, about 2 to about 12 wt. %, about 2 to about 10 wt. %, about 2 to about 8 wt. %, about 2 to about 6 wt. %; from about 6 to about 20 wt. %, about 6 to about 16 wt. %, about 6 to about 12 wt. %, about 6 to about 10 wt. %; from about 8 to about 20 wt. %, about 8 to about 16 wt. %, about 8 to about 12 wt. %, about 8 to about 10 wt. %; from about 10 to about 20 wt. %, about 10 to about 16 wt. %; from about 12 to about 20 wt. %, about 12 to about 16 wt. %; from about 14 to about 20 wt. %, or about 14 to about 16 wt. %, including ranges and subranges thereof, based on the total weight of the dog food composition on a dry matter basis.

Dietary fiber refers to components of a plant which are resistant to digestion by an animal's digestive enzymes. Dietary fiber includes soluble fiber and insoluble fiber. Soluble fibers are resistant to digestion and absorption in the small intestine and undergo complete or partial fermentation in the large intestine, e.g., beet pulp, guar gum, chicory root, psyllium, pectin, blueberry, cranberry, squash, apples, oats, beans, citrus, barley, or peas. Insoluble fibers can be supplied by any of a variety of sources, including, for example, pecan shell, buckwheat hull, cellulose, whole-wheat products, wheat oat, corn bran, flax seed, grapes, celery, green beans, cauliflower, potato skins, fruit skins, vegetable skins, peanut hulls, and soy fiber. Crude fiber includes indigestible components contained in cell walls and cell contents of plants such as grains, for example, hulls of grains such as rice, corn, and beans. Typical crude fiber amounts in compositions of the present disclosure can be from about 0 to about 20 wt. %, about 1 to about 20 wt. %, about 1 to about 15 wt. %, about 1 to about 10 wt. %, about 1 to about 7 wt. %, about 1 to about 5 wt. %, about 1 to about 3 wt. %; from about 3 to about 20 wt. %, about 3 to about 15 wt. %, about 3 to about 10 wt. %, about 3 to about 7 wt. %, about 3 to about 5 wt. %; from about 5 to about 20 wt. %, about 5 to about 15 wt. %, about 5 to about 10 wt. %, about 5 to about 7 wt. %; from about 7 to about 20 wt. %, about 7 to about 15 wt. %, or about 7 to about 10 wt. %, including any ranges or subranges thereof, based on the total weight of the dog food composition on a dry matter basis.

The dog food composition may include from about 1 to about 9 wt. % of insoluble fiber, based on the total weight of the dog food composition on a dry matter basis. For example, the amount of insoluble fiber present in the dog food composition may be from about 1 to about 9 wt. %, about 1 to about 8 wt. %, about 1 to about 7 wt. %, about 1 to about 6 wt. %, about 1 to about 5 wt. %, about 1 to about 4 wt. %, about 1 to about 3 wt. %; from about 2 to about 9 wt. %, about 2 to about 8 wt. %, about 2 to about 7 wt. %, about 2 to about 6 wt. %, about 2 to about 5 wt. %, about 2 to about 4 wt. %; from about 3 to about 9 wt. %, about 3 to about 8 wt. %, about 3 to about 7 wt. %, about 3 to about 6 wt. %, about 3 to about 5 wt. %; from about 4 to about 9 wt. %, about 4 to about 8 wt. %, about 4 to about 7 wt. %, or about 4 to about 6 wt. %, including ranges and subranges thereof, based on the total weight of the dog food composition on a dry matter basis.

The dog food composition may include from about 0.2 to about 4 wt. % of soluble fiber, based on the total weight of the dog food composition on a dry matter basis. For example, amount of soluble fiber present in the dog food composition may be from about 0.2 to about 4 wt. %, about 0.2 to about 3 wt. %, about 0.2 to about 2 wt. %, about 0.2 to about 1 wt. %; from about 0.5 to about 4 wt. %, about 0.5 to about 3 wt. %, about 0.5 to about 2 wt. %, about 0.5 to about 1 wt. %; from about 0.75 to about 4 wt. %, about 0.75 to about 3 wt. %, about 0.75 to about 2 wt. %; from about 1 to about 4 wt. %, about 1 to about 3 wt. %, about 1 to about 2 wt. %; from about 2 to about 4 wt. %, or about 2 to about 3 wt. %, including ranges and subranges thereof, based on the total weight of the dog food composition on a dry matter basis.

The dog food composition may be formulated to have a weight ratio of soluble fiber to insoluble fiber of from about 1:20 to about 10:1. For example, the dog food composition may have a weight ratio of soluble fiber to insoluble fiber of from about 1:18 to about 5:1, or about 1:18 to about 2:1, or about 1:18 to about 1:1, or about 1:18 to about 1:2.

The dog food composition typically comprise protein and/or a digestible crude protein. The term “protein” means a polypeptide, or a peptide, or a polymer of amino acids. The term encompasses naturally occurring and non-naturally occurring (synthetic) polymers and polymers in which artificial chemical mimetics are substituted for one or more amino acids. The term also encompasses fragments, variants, and homologs that have the same or substantially the same properties and perform the same or substantially the same function as the original sequence. The term encompasses polymers of any length, including polymers containing from about 2 to 1000, from 4 to 800, from 6 to 600, and from 8 to 400 amino acids. The protein may include amino acid polymers that are synthesized and that are isolated and purified from natural sources. Under some aspects, the terms “polypeptide”, “peptide” or “protein” are used interchangeably.

“Digestible crude protein” is the portion of protein that is available or can be converted into free nitrogen (amino acids) after digesting with gastric enzymes. In vitro measurement of digestible crude protein may be accomplished by using gastric enzymes such as pepsin and digesting a sample and measuring the free amino acid after digestion. In vivo measurement of digestible crude protein may be accomplished by measuring the protein levels in a feed/food sample and feeding the sample to an animal and measuring the amount of nitrogen collected in the animal's feces.

The protein and/or digestible crude protein of the composition may be present at various amounts or concentrations. In one aspect, protein may be present in an amount of from about 10 to about 40 wt. %, based on the total weight of the dog food composition on a dry matter basis. For example, protein may be present in an amount of from about 10 to about 40 wt. %, about 10 to about 36 wt. %, about 10 to about 32 wt. %, about 10 to about 28 wt. %, about 10 to about 24 wt. %, about 10 to about 20 wt. %, about 10 to about 18 wt. %; from about 12 to about 40 wt. %, about 12 to about 36 wt. %, about 12 to about 32 wt. %, about 12 to about 28 wt. %, about 12 to about 24 wt. %, about 12 to about 20 wt. %, about 12 to about 18 wt. %; from about 14 to about 40 wt. %, about 14 to about 36 wt. %, about 14 to about 32 wt. %, about 14 to about 28 wt. %, about 14 to about 24 wt. %, about 14 to about 20 wt. %, about 14 to about 18 wt. %; from about 16 to about 40 wt. %, about 16 to about 36 wt. %, about 16 to about 32 wt. %, about 16 to about 28 wt. %, about 16 to about 24 wt. %, about 16 to about 20 wt. %, or about 16 to about 18 wt. %, including ranges or subranges thereof, based on the total weight of the dog food composition on a dry matter basis. In some aspects, the dog food composition includes protein in amount of about 10 wt. %, about 15 wt. %, about 20 wt. %, about 25 wt. %, about 30 wt. %, about 35 wt. %, about 40 wt. %, or any range formed therefrom, based on the total weight of the dog food composition on a dry matter basis. In another example, protein may be present in an amount of from about 10 to about 25 wt. %, about 15 to about 25 wt. %, or about 15 to about 20 wt. %, based on the total weight of the dog food composition on a dry matter basis on a dry matter basis. In certain aspects, protein is present in an amount of about 12 to about 35 wt. %, about 13 to about 25 wt. %, or about 15 to about 25 wt. %, based on the total weight of the dog food composition on a dry matter basis.

A portion of the protein in the composition may be digestible protein. For example, the composition may include an amount of protein, where about 40 wt. % or more, about 50 wt. % or more, about 60 wt. % or more, about 70 wt. % or more, about 80 wt. % or more, about 90 wt. % or more, about 95 wt. % or more, about 98 wt. % or more, or about 99 wt. % or more of the protein is digestible protein. In some aspects, e.g., when the composition desirable promotes weight loss, the portion of protein that is digestible protein is about 60 wt. % or less, about 50 wt. % or less, about 40 wt. % or less, about 30 wt. % or less, about 20 wt. % or less, or about 10 wt. % or less, based on the total amount of protein in the dog food composition on a dry matter basis. In further aspect, the amount of protein that is digestible protein is about 10 to about 99 wt. %, about 10 to about 95 wt. %, about 10 to about 90 wt. %, about 10 to about 70 wt. %, about 10 to about 50 wt. %, about 10 to about 30 wt. %; about 30 to about 99 wt. %, about 30 to about 95 wt. %, about 30 to about 90 wt. %, about 30 to about 70 wt. %, about 30 to about 50 wt. %; about 50 to about 99 wt. %, about 50 to about 95 wt. %, about 50 to about 90 wt. %, about 50 to about 70 wt. %; or about 70 to about 99 wt. %, about 70 to about 95 wt. %, about 70 to about 90 wt. %, including ranges and subranges therein, based on the total amount of protein in the dog food composition on a dry matter basis.

Protein may be supplied by any of a variety of sources known by those of ordinary skill in the art including plant sources, animal sources, microbial sources or a combination of these. For example, animal sources may include meat, meat-by products, seafood, dairy, eggs, etc. Meats, for example, may include animal flesh such as poultry, fish, and mammals including cattle, pigs, sheep, goats, and the like. Meat by-products may include, for example, lungs, kidneys, brain, livers, stomachs and intestines. Plant protein includes, for example, soybean, cottonseed, and peanuts. Microbial sources may be used to synthesize amino acids (e.g., lysine, threonine, tryptophan, methionine) or intact protein such as protein from sources listed below.

Examples of protein or protein ingredients may comprise chicken meals, chicken, chicken by-product meals, lamb, lamb meals, turkey, turkey meals, beef, beef by-products, viscera, fish meal, enterals, kangaroo, white fish, venison, soybean meal, soy protein isolate, soy protein concentrate, corn gluten meal, corn protein concentrate, distillers dried grains, and/or distillers dried grain solubles and single-cell proteins, for example yeast, algae, and/or bacteria cultures.

The protein can be intact, completely hydrolyzed, or partially hydrolyzed. The protein content of foods may be determined by any number of methods known by those of skill in the art, for example, as published by the Association of Official Analytical Chemists in Official Methods of Analysis (“OMA”), method 988.05. The amount of protein in a composition disclosed herein may be determined based on the amount of nitrogen in the composition according to methods familiar to one of skill in the art.

The compositions of the present invention may optionally comprise fat. The term “fat” generally refers to a lipid or mixture of lipids that may generally be a solid or a liquid at ordinary room temperatures (e.g., 25° C.) and pressures (e.g., 1 atm). In some instances, the fat may be a viscous liquid or an amorphous solid at standard room temperature and pressure. The fat may be incorporated completely within the food composition, deposited on the outside of the dog food composition, or a mixture of the two methods. In some aspects, the dog food compositions further include an effective amount of one or more substances selected from the group consisting of glucosamine, chondroitin, chondroitin sulfate, methylsulfonylmethane (“MSM”), creatine, antioxidants, Perna canaliculata, and mixtures thereof.

Fat can be supplied by any of a variety of sources known by those skilled in the art, including meat, meat by-products, canola oil, fish oil such as anchovy oil and menhaden oil, and plants. Meat fat sources include poultry fat, turkey fat, pork fat, lard, tallow, and beef fat. Plant fat sources include wheat, flaxseed, rye, barley, rice, sorghum, corn, oats, millet, wheat germ, corn germ, soybeans, peanuts, and cottonseed, as well as oils derived from these and other plant fat sources such as corn oil, soybean oil, cottonseed oil, palm oil, palm kernel oil, linseed oil, canola oil, rapeseed oil, and/or olestra.

In some cases, the fat in the compositions is crude fat. Crude fat may be included into the compositions in the amounts disclose above with respect of the total fat, such as 10 wt. %, based on the total weight of the dog food composition on a dry matter basis. Alternatively, about 50 wt. % or more, about 60 wt. % or more, about 70 wt. % or more, about 80 wt. % or more, or about 90 wt. % or more of the total fat may be obtained from a plant source.

The term “carbohydrate” as used herein includes polysaccharides (e.g., starches and dextrins) and sugars (e.g., sucrose, lactose, maltose, glucose, and fructose) that are metabolized for energy when hydrolyzed. One skilled in the art could manipulate the texture of the final product by properly balancing carbohydrate sources. For example, short chain polysaccharides lend to be sticky and glucy, and longer chain polysaccharides are less sticky and gluey than the shorter chain; the desired texture of this hybrid food is achieved by longer chain polysaccharide and modified starches such as native or modified starches, cellulose and the like. The carbohydrate mixture may additionally comprise optional components such as added salt, spices, seasonings, vitamins, minerals, flavorants, colorants, and the like. The amount of the optional components is at least partially dependent on the nutritional requirements for different life stages of animals.

Carbohydrates can be supplied by any of a variety of sources known by those skilled in the art, including, but not limited to, oat fiber, cellulose, peanut hulls, beet pulp, parboiled rice, corn starch, corn gluten meal, cereal, and sorghum. Grains supplying carbohydrates can include, but are not limited to, wheat, durum, semolina, corn, barley, and rice. In certain aspects, the carbohydrate component comprises a mixture of one or more carbohydrate sources. Carbohydrates content of foods can be determined by any number of methods known by those of skill in the art.

Generally, carbohydrate percentage can be calculated as nitrogen free extract (“NFE”), which can be calculated as follows: NFE=100%−(moisture %)−(protein %)−(fat %)−(ash %)−(crude fiber %). The amount of carbohydrate, e.g., calculated as NFE, present in the composition may be about 10 to about 90 wt. %, about 10 to about 70 wt. %, about 10 to about 50 wt. %, about 10 to about 40 weight %, about 10 to about 30 wt. %, about 10 to about 20 wt. %; about 20 to about 90 wt. %, about 20 to about 70 wt. %, about 20 to about 50 wt. %, about 20 to about 40 weight %; about 30 to about 90 wt. %, about 30 to about 70 wt. %, about 30 to about 50 wt. %, about 30 to about 40 weight %; about 50 to about 90 wt. %, about 50 to about 70 wt. %; or about 70 to about 90 wt. %, based on the total weight of the dog composition on a dry matter basis.

In certain aspects, the dog food composition comprises moisture. The moisture may be present at various amounts or concentrations. In one aspect, moisture may be present in an amount of from about 3 to about 20 wt. %, based on the total weight of the dog food composition. For example, moisture may be present in an amount of about 3 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, about 10.5 wt. %, about 11 wt. %, about 11.5 wt. %, about 12 wt. %, about 12.5 wt. %, about 13 wt. %, about 13.5 wt. %, about 14 wt. %, about 14.5 wt. %, or about 15 wt. %, based on the total weight of the dog food composition. In another example, moisture may be present in an amount of from about 6% to about 12%, about 9% to about 13%, about 9% to about 11%, or about 9% to about 13%, based on the total weight of the dog food composition. In certain aspects, moisture is present in an amount of about 5% to about 12%, about 6% to about 11%, or about 7% to about 10.0%, based on the total weight of the dog food composition. In further aspects, moisture is present in an amount of about 65% to about 85%, about 60% to about 80%, or about 60% to about 75%, based on the total weight of the dog food composition.

The dog food compositions may include one or more ingredients and/or sources of glucose mimetic, carotenoids, and/or arginine and derivatives thereof. Sources of glucose mimetics may comprise glucose anti-metabolites including 2-deoxy-D-glucose, 5-thio-D-glucose, 3-O-methylglucose, anhydrosugars including 1,5-anhydro-D-glucitol, 2,5-anhydro-D-glucitol, and 2,5-anhydro-D-mannitol, mannoheptulose, and/or avocado extract comprising mannoheptulose. Sources of carotenoids may include lutein, astaxanthin, zeaxanthin, bixin, lycopene, and/or beta-carotene. Sources of antioxidant ingredients may comprise tocopherols (vitamin E), vitamin C, vitamin A, plant-derived materials, carotenoids (described above), selenium, and/or CoQ10 (Co-enzyme Q10). In a preferred aspect, the dog food composition contains high levels of arginine and derivatives thereof. The amount of arginine present in the composition may be about 0.01 to about 10.0 wt. %, about 0.01 to about 5.0 wt. %, about 0.01 to about 2.0 wt. %, about 0.1 to about 10.0 wt. %, about 0.1 to about 5.0 wt. %, about 0.1 to about 2.0 wt. %; about 0.5 to about 5.0 wt. %, about 0.5 to about 2.0 wt. %, about 1.5 to about 5.0 wt. %, about 1.5 to about 2.0 wt. %, about 0.5 wt. %, about 1.0 wt. %, about 1.4 weight %, about 1.44 wt. %, about 1.8 wt. %, or about 2.0 wt. %, based on the total weight of the composition on a dry matter basis. The arginine present in the composition may L-arginine, D-arginine, or a mixture thereof.

The dog food compositions disclosed herein may be wet or dry compositions, and the ingredients can be either incorporated into the food composition and/or on the surface of any composition component, such as, for example, by spraying, agglomerating, dusting, or precipitating on the surface. Additionally, the dog food compositions may be formulated and produced to be in various forms and/or consistencies. For instance, the dog food compositions may, for example, be a dry, moist or semi-moist animal food composition. “Semi-moist” refers to a food composition containing from about 25 to about 35% moisture. “Moist” food refers to a food composition that has a moisture content of about 60 to 90% or greater. “Dry” food refers to a food composition with about 3 to about 12% moisture content and is often manufactured in the form of small bits or kibbles.

The food products may also include components of more than one consistency, for example, soft, chewy meat-like particles or pieces as well as kibble having an outer coating and an inner “core” component. In some aspects, the dog food compositions may be in the form of a kibble or food kibble. As used herein, the term “kibble” or “food kibble” refers to a particulate pellet, e.g., like a component of or canine feeds. In some aspects, a food kibble has a moisture, or water, content of less than 15% by weight. Food kibbles may range in texture from hard to soft. Food kibbles may range in internal structure from expanded to dense. Food kibbles may be formed by an extrusion process or a baking process. In non-limiting examples, a food kibble may have a uniform internal structure or a varied internal structure. For example, a food kibble may include a core and a coating to form a coated kibble. It should be understood that when the term “kibble” or “food kibble” is used, it can refer to an uncoated kibble or a coated kibble.

In accordance with one aspect of the disclosure, the dog food composition is in a form comprising a core and a coating disposed on the core. In one aspect, the core comprises an alpha-amino acid (e.g., lysine) and one or more ancient grain (e.g., quinoa and an amaranth), while the coating comprises eggshell membrane.

The dog food composition may comprise a binder, for example, to retain the kibble form and/or to bind the out coating to the core. In certain aspects the binder includes but is not limited to any of the following or combinations of the following: monosaccharides such as glucose, fructose, mannose, arabinose; di- and trisaccharides such as sucrose, lactose, maltose, trehalose, lactulose; corn and rice syrup solids; dextrins such as corn, wheat, rice and tapioca dextrins; maltodextrins; starches such as rice, wheat, corn, potato, tapioca starches, or these starches modified by chemical modification; alginates, chitosans; gums such as carrageen, and gum arabic; polyols such as glycerol, sorbitol, mannitol, xylitol, erythritol; esters of polyols such as sucrose esters, polyglycol esters, glycerol esters, polyglycerol esters, sorbitan esters; sorbitol; molasses; honey; gelatins; peptides; proteins and modified proteins such as whey liquid, whey powder, whey concentrate, whey isolate, whey protein isolate, high lactose whey by-product, meat broth solids such as chicken broth, chicken broth solids, soy protein, and egg white. In one aspect, the dog food composition is substantially free of or free of egg white and/or whole egg ingredients.

In certain aspects, the binder includes but is not limited to a lipid and/or lipid derivative. Lipids can be used in combination with water and/or other binder components. Lipids can include plant fats such as soybean oil, corn oil, rapeseed oil, olive oil, safflower oil, palm oil, coconut oil, palm kernel oil, and partially and fully hydrogenated derivatives thereof; animal fats and partially and fully hydrogenated derivatives thereof; and waxes.

The composition of the present disclosure can additionally comprise other additives in amounts and combinations familiar to one of skill in the art. Such additives should be present in amounts that do not impair the purpose and effect provided by the invention. Examples of additives include substances with a stabilizing effect, organoleptic substances, processing aids, and substances that provide nutritional benefits.

Stabilizing substances may include, by way of example, substances that tend to increase the shelf life of the composition. Other examples of other such additives potentially suitable for inclusion in the compositions of the invention include, for example, preservatives, antioxidants, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Additives for coloring, palatability, and nutritional purposes can include colorants, salts (including, but not limited to, sodium chloride, potassium citrate, potassium chloride, and other edible salts), vitamins, minerals, and flavoring. Other additives can include glucosamine, chondroitin sulfate, vegetable extracts, herbal extracts, etc.

The concentration of such additives in the composition typically can be up to about 5% by weight, based on the total weight of the dog food composition on a dry matter basis. In some aspects, the concentration of such additives (particularly where such additives are primarily nutritional balancing agents, such as vitamins and minerals) is from about 0% to about 2.0% by weight, based on the total weight of the dog food composition on a dry matter basis. In some aspects, the concentration of such additives (again, particularly where such additives are primarily nutritional balancing agents) is from about 0% to about 1.0% by weight, based on the total weight of the dog food composition on a dry matter basis. Although the list of foregoing additives may be potentially suitable in some aspects, one or more of the foregoing additives may be excluded from other aspects of the dog food composition.

The dog food composition may be produced by various methods to achieve the desired dog food composition or desired form for the dog food composition. For example, dry food may be baked or extruded, then cut into individual shaped portions, such as kibbles. In some aspects, the dog food composition may be prepared in a canned or wet form using conventional food preparation processes known to those of ordinary skill in the art. Typically, ground animal proteinaceous tissues are mixed with the other ingredients, such as cercal grains, suitable carbohydrate sources, fats, oils, and balancing ingredients, including special purpose additives such as vitamin and mineral mixtures, inorganic salts, cellulose, beet pulp and the like, and water in an amount sufficient for processing. The ingredients are mixed in a vessel suitable for heating while blending the components. Heating the mixture is carried out using any suitable manner, for example, direct steam injection or using a vessel fitted with a heat exchanger. Following addition of all of the ingredients of the formulation, the mixture may be heated to a temperature of from 50° F. to 212° F. Although temperatures outside this range can be used, they may be commercially-impractical without the use of other processing aids. When heated to the appropriate temperature, the material will typically be in the form of thick liquid, which is dispensed into cans. A lid is applied and the container is hermetically sealed. The sealed can is then placed in convention equipment designed for sterilization of the contents. Sterilization is usually accomplished by heating to temperatures of greater than 230° C. for an appropriate time depending on the temperature used, the nature of the composition, and related factors. The dog food compositions and food products of the present disclosure can also be added to or combined with food compositions before, during, or after their preparation.

In some aspects, the food products may be prepared in a dry form using convention processes known to those of ordinary skill in the art. Typically, dry ingredients, including dried animal protein, plant protein, grains and the like are ground and mixed together. Liquid or moist ingredients, including fats, oils water, animal protein, water, and the like are added combined with the dry materials. The specific formulation, order of addition, combination, and methods and equipment used to combine the various ingredients can be selected from those known in the art. For example, in certain aspects, the resulting mixture is process into kibbles or similar dry pieces, which are formed using an extrusion process in which the mixture of dry and wet ingredients is subjected to mechanical work at high pressure and temperature, forced through small openings or apertures, and cut off into the kibbles, e.g., with a rotating knife. The resulting kibble can be dried and optionally coated with one or more topical coatings comprising, e.g., flavors, fats, oils, powdered ingredients, and the like. Kibbles may also be prepared from dough by baking, rather than extrusion, in which the dough is placed into a mold before dry-heat processing.

In preparing a composition, any ingredient generally may be incorporated into the composition during the processing of the formulation, e.g., during and/or after mixing of the other components of the composition. Distribution of these components into the composition can be accomplished by conventional means. In certain aspects, ground animal and/or poultry proteinaceous tissues are mixed with other ingredients, including nutritional balancing agents, inorganic salts, and may further include cellulose, beet pulp, bulking agents and the like, along with sufficient water for processing.

### EXAMPLES

The following Examples are provided to further describe certain preferred embodiments of the invention and are not to be construed to limit the scope of the invention.

### Example 1

Feces samples were collected from 24 dogs of varying age, metabolic rate, gender, body weight, feces score GI Phenotype and health status at time of sample collection. See, Table 1, below.

TABLE 1
Characteristics of dogs used in the study

| | | | | | | | | Specific Health |
|---|---|---|---|---|---|---|---|---|
| | | | | | | | | Problem |
| | Animal Name- | Metabolic | | | BW | Feces | GI | At Sample |
| Sample No. | Tattoo | Rate | Age | Sex | (Kg) | Score | Phenotype | Collection |
| 50092693.0025 | BESS-4481895 | 7 | 11Y, 11M | SF | 10.2 | 4 | No GI risk | Healthy/Retired |
| 50093131.0025 | BlllZEN-1YX-1 | 10 | 3Y, 3M | NM | 11.6 | 3 | No GI risk | Healthy |
| 50092695.0025 | HAMLIN-5438870 | 3 | 8Y, 1M | NM | 11.8 | 4 | No GI risk | Healthy |
| 50092689.0025 | HARD ROCK-1971702 | 4 | 3Y, 0M | NM | 11.2 | 5 | No GI risk | Healthy |
| 50092696.0025 | MCMURRAY-5444136 | 6 | 8Y, 1M | NM | 11.5 | 5 | No GI risk | Healthy |
| 50092694.0025 | OMAHA-4975286 | 4 | 9Y, 7M | SF | 10.5 | 4 | No GI risk | Healthy |
| 50092690.0025 | PHELPS-088AC | 7 | 6Y, 6M | NM | 9.7 | 5 | No GI risk | Healthy |
| 50094175.0025 | RAINE-H1F057 | 8 | 14Y, 5M | SF | 11.6 | 3 | No GI risk | Healthy/aged |
| 50092687.0025 | TARZAN-L0581603 | 8 | 10Y, 1M | NM | 11.9 | 4 | No GI risk | Healthy |
| 50093204.0025 | WYNN-1973062 | 7 | 3Y, 1M | SF | 11.8 | 3 | No GI risk | Healthy/pregnant |
| 50092697.0025 | ANGELINA-5438560 | 5 | 8Y, 2M | SF | 7.8 | 4 | GI risk | Acute Colitis |
| | | | | | | | | with blood |
| 50092688.0025 | AQUATA-FGS-1 | 10 | 3Y, 6M | SF | 8.2 | 5 | GI risk | Dermatitis |
| 50093380.0025 | AQUATA-FGS-1 | 10 | 3Y, 6M | SF | 8.2 | 2 | GI risk | Dermatitis |
| 50093205.0025 | BALTIMORE-4965256 | 6 | 9Y, 8M | NM | 10.4 | 2 | GI risk | Loose stool/ |
| | | | | | | | | skin problems |
| 50092692.0025 | BOWSER-CTSABS | 4 | 12Y, 11M | NM | 13.6 | 5 | GI risk | Food allergy/ |
| | | | | | | | | dermatitis |
| 50094384.0025 | BOWSER-CTSABS | 4 | 12Y, 10M | NM | 12.9 | 1 | GI risk | Food allergy/ |
| | | | | | | | | dermatitis |
| 50093206.0025 | BURGER BEAGLE- | 4 | 3Y, 6M | NM | 10.8 | 1 | GI risk | IBD |
| | XTT-1 | | | | | | | |
| 50093201.0025 | BURGER BEAGLE- | 4 | 3Y, 6M | NM | 10.6 | 2 | GI risk | IBD |
| | XTT-1 | | | | | | | |
| 50093890.0025 | CONNOR-H2B046 | 8 | 13Y, 7M | NM | 11.9 | 2 | GI risk | Loose stools/ |
| | | | | | | | | skin problems |
| 50093132.0025 | DAKOTA-H2B044 | 9 | 13Y, 3M | NM | 9.7 | 3 | GI risk | Adenocarcinoma |
| 50093199.0025 | MARLIN-5443580 | 6 | 8Y, 2M | NM | 10.3 | 1 | GI risk | Acute colitis with |
| | | | | | | | | blood stool/lBD |
| 50093196.0025 | SAHARA-1972724 | 6 | 3Y, 0 M | SF | 9.4 | 3 | GI risk | Dermatitis diag: |
| | | | | | | | | June |
| | | | | | | | | 2013 |
| 50095335.0025 | SANTA-VFZ-1 | 7 | 3Y, 4 M | NM | 16 | 1 | GI risk | IBD |
| 50093133.0025 | STINKY PETE-H2B002 | 8 | 14Y, 1M | NM | 17.7 | 2 | GI risk | Aggressiveness/ |
| | | | | | | | | hypothyroidism |

(SF = spayed female; NM = neutered male; BW = body weight; GI = gastrointestinal)

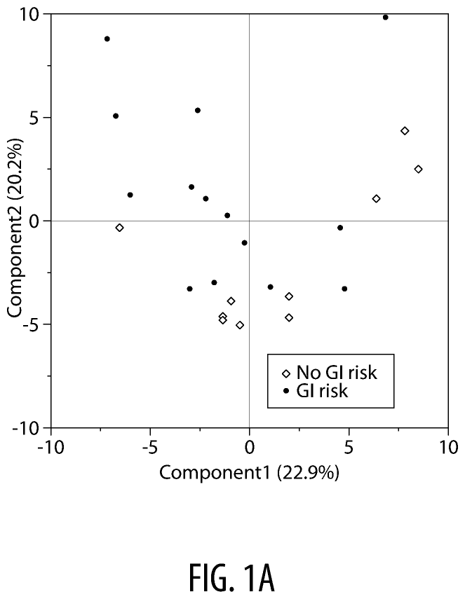

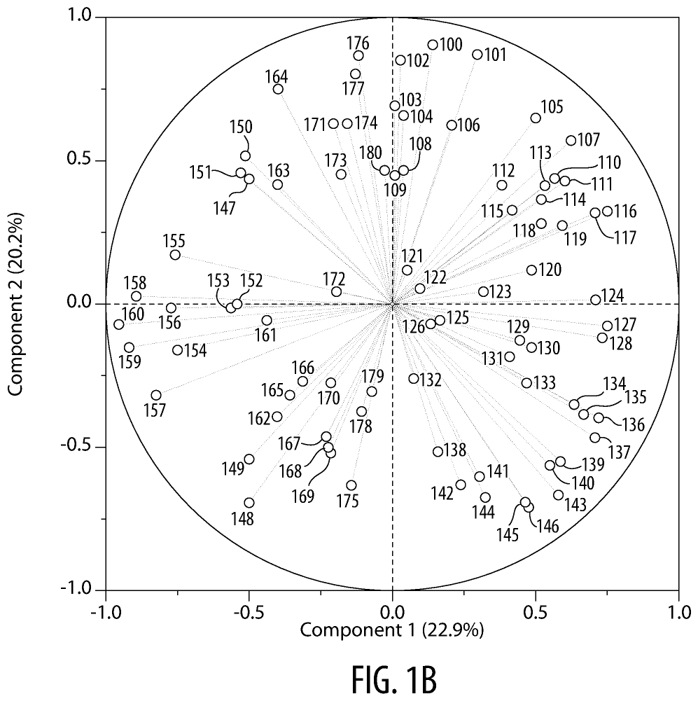

Data obtained from 24 fecal samples, including microbiome data at three levels (genera (g), Family (f), phyla (p)), SCFA, pH and associated metadata (a total of 96 variables), were included in PCA analysis and it was observed that there is segregation between the “GI risk” group vs “No GI risk” group with more overlapping (FIGS. 1A and 1B). Specifically, the components one and two explained only 22.9% and 20.2% variation, respectively. Evaluation of the 96 variables between “GI risk” vs “No GI risk” by PLSDA resulted in 16 candidate biomarkers (see, Table 2, below). Variables marked with asterisks were positively associated and variables marked with number signs were negatively associated with group ‘No GI risk”. Bold entries, Total SCFA through f_Enterobacteriaceae, indicate the marked variable's importance as contributors in distinguishing the groups “No GI risk” vs “GI risk” based on VIP scores. Model qualities: R2 cumulative: 0.537 and Q2 cumulative: 0.472. In FIG. 1B, the numbering substitutes for labels, as follows:

TABLE 1

| | |
|---|---|
| 100 | f. clostridiaceae |
| 101 | g. clostridium |
| 102 | g. SMB53 |
| 103 | f. enterobacteriaceae |
| 104 | not labeled |
| 105 | f. lachnospiraceae |
| 106 | not labeled |
| 107 | f. erysipelotrichaceae |
| 108 | g. catenibacterium |
| 109 | f. Paraprevotellaceae |
| 110 | g. blautia |
| 111 | not labeled |
| 112 | f. paraprevotellaceae |
| 113 | not labeled |
| 114 | not labeled |
| 115 | p. proteobacteria |
| 116 | g. megamonas |
| 117 | g. megamonas |
| 118 | f. veillonellaceae |
| 119 | C2:C4 |
| 120 | g. slackia |
| 121 | BW |
| 122 | f. alcaligenaceae |
| 123 | f. fusobacteriaceae |
| 124 | not labeled |
| 125 | g. dialister |
| 126 | p. fusobacteria |
| 127 | f. succinivibrionaceae |
| 128 | f. paraprevotellaceae |
| 129 | not labeled |
| 130 | not labeled |
| 131 | f. ruminococcaceae |
| 132 | C5, ppm |
| 133 | g. roseburia |
| 134 | g51.Evenness |
| 135 | f. bacteroidaceae |
| 136 | g. phascolarctobacterium |
| 137 | f. succinivibrionaceae |
| 138 | feces score |
| 139 | G49.Diversity |
| 140 | not labeled |
| 141 | TotalSCFA |
| 142 | C2, ppm |
| 143 | p. bacteroidetes |
| 144 | f. prevotellaceae |
| 145 | g. prevotella |
| 146 | f. prevotellaceae |
| 147 | pH |
| 148 | f. S24-7 |
| 149 | f. mogibacteriaceae |
| 150 | f. clostridiaceae |
| 151 | f. erysipelotrichaceae |
| 152 | o. clostridiales |
| 153 | not labeled |
| 154 | g. bidobacterium |
| 155 | p. actinobacteria |
| 156 | not labeled |
| 157 | f. turicibacteriaceae |
| 158 | not labeled |
| 159 | not labeled |
| 160 | g. allocbaculum |
| 161 | g. lactobacillus |
| 162 | f. pepptococcaceae |
| 163 | g. streptococcus |
| 164 | f. peptostreptococcaceae |
| 165 | f. turicibacteraceae |
| 166 | o. clostridiales |
| 167 | Sum2mC3_C4 |
| 168 | 2mC4, ppm |
| 169 | 2mC3, ppm |
| 170 | G50.Richness |
| 171 | f. coriobacteriaceae |
| 172 | Metabolic.Rate |
| 173 | g. streptococcus |
| 174 | g. ruminococcus |
| 175 | C4, ppm |
| 176 | c. clostridiaceae |
| 177 | p. peptostreptococcaceae |
| 178 | G50.Richness |
| 179 | C6, ppm |
| 180 | g. streptococcus |

TABLE 2
Features identified by PLS-DA and their variable
importance of projection (VIP) scores.

| Variable | VIP | Coeff-Healthy |
|---|---|---|
| Total SCFA*** | 1.473 | 0.0000082615494 |
| C3, ppm*** | 1.386 | 0.0000226313371 |
| C2, ppm*** | 1.372 | 0.0000144835243 |
| pH^{###} | 1.302 | −0.0708171163708 |
| Feces Score*** | 1.129 | 0.0272064550332 |
| Moisture^{###} | 1.110 | −0.5010666143704 |
| f_{—}Prevotellaceae*** | 1.091 | 0.0166955942986 |
| f_{—}Enterobacteriaceae^{###} | 1.072 | −0.0124090997087 |
| C4, ppm*** | 0.949 | 0.0000241826666 |
| f_Ruminococcaceae*** | 0.864 | 0.0333004720446 |
| Others^{###} | 0.794 | −0.0306461125956 |
| C5, ppm*** | 0.744 | 0.0000520928989 |
| C6, ppm*** | 0.582 | 0.0009228285880 |
| f_Bacteroidaceae*** | 0.506 | 0.0122202335246 |
| p_Bacteroidetes-F3&4^{###} | 0.227 | −0.0105901500945 |
| O_Clostridiales; f_{——}*** | 0.088 | 0.0023098743950 |

***= positively associated with No Risk;
^{###}= negatively associated with No Risk.
Bold letters indicate importance as contributors in distinguishing the groups “No GI risk” vs “GI risk” based on VIP scores. Model qualities: R2 cumulative: 0.537 and Q2 cumulative: 0.472.

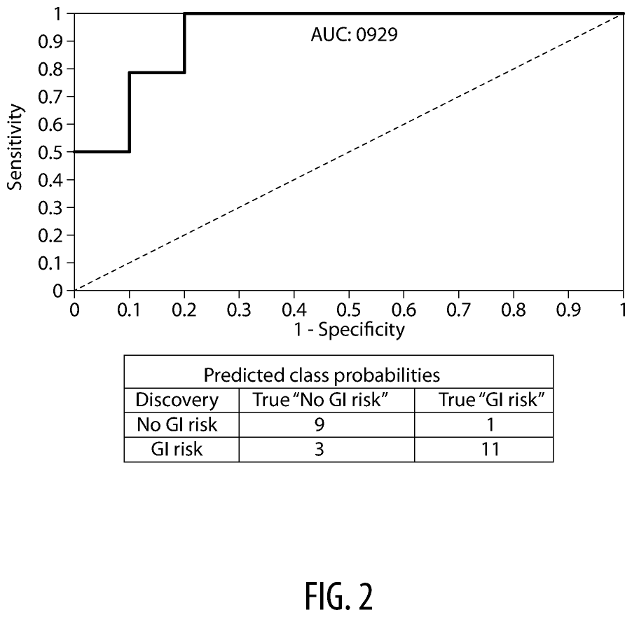

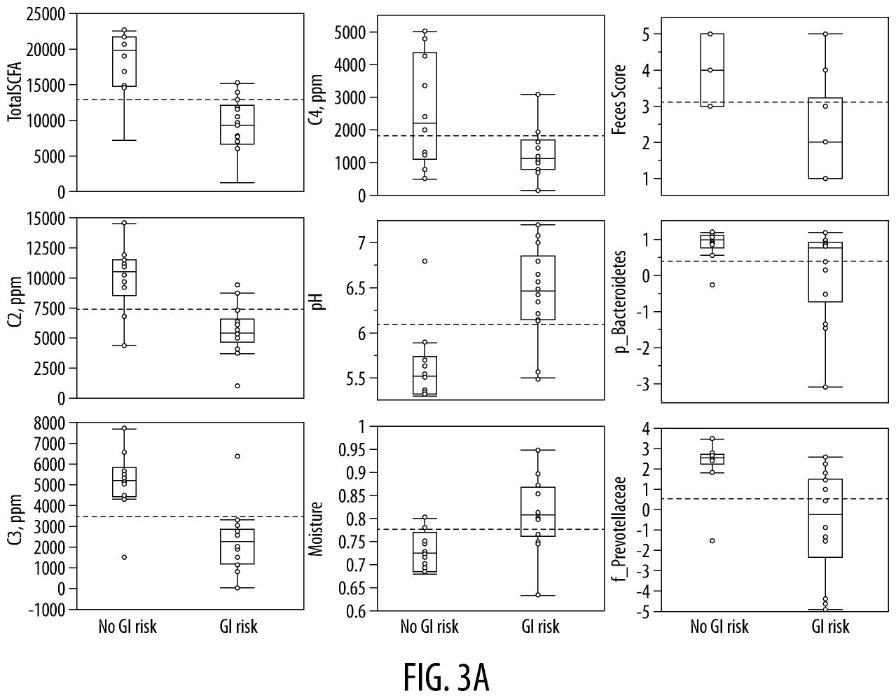

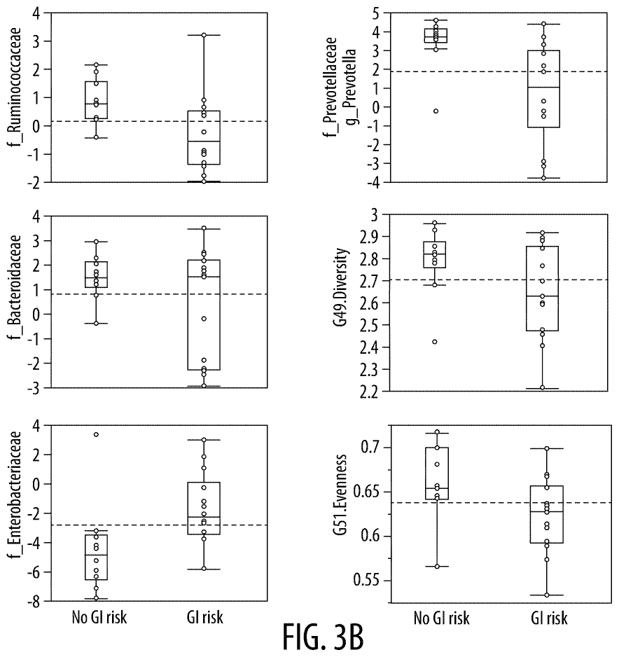

This panel of 16 biomarkers differentiated subjects with “GI risk” from “No GI risk” with an AUC of 0.929 and a classification accuracy of 0.83 (FIG. 2). Notably, the relative abundance of 15 variables (Total SCFA, C2, C3, C4, pH, moisture, fecal score, Phylum Bacteroidetes-excluding F3: f_Bacteroidaceae and F4: f_Prevotellaceae, families Prevotellaceae, Ruminococcaceae, Enterobacteriaceae, Bacteroidaceae, genus Prevotella, Shannon diversity and evenness) was significantly different (based on Wilcoxon/Kruskas-Wallis test, p<0.05) between “GI risk” vs “No GI risk” (FIG. 3).

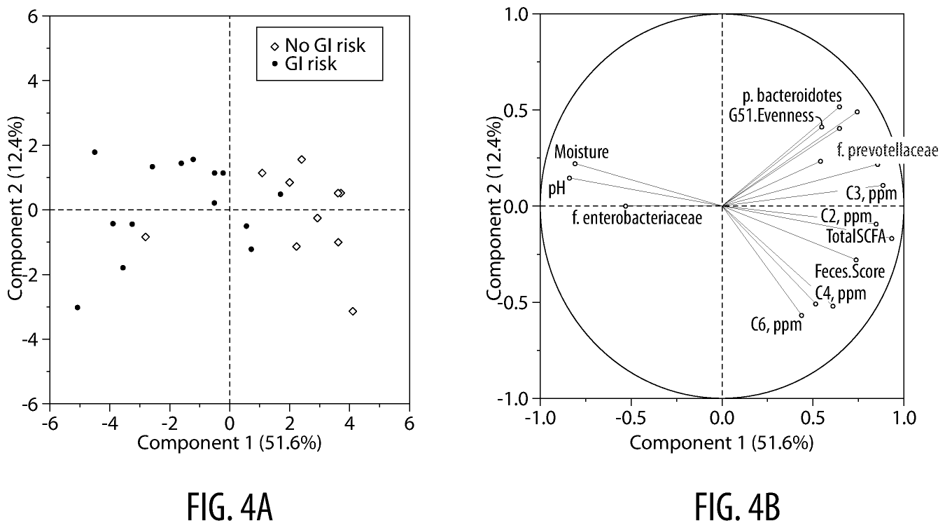

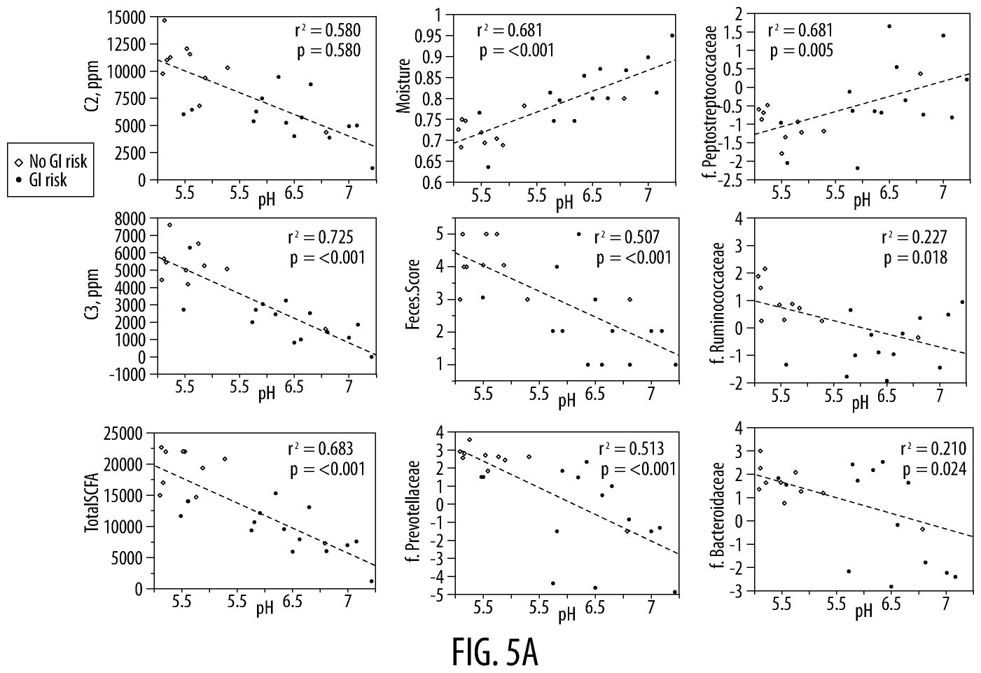

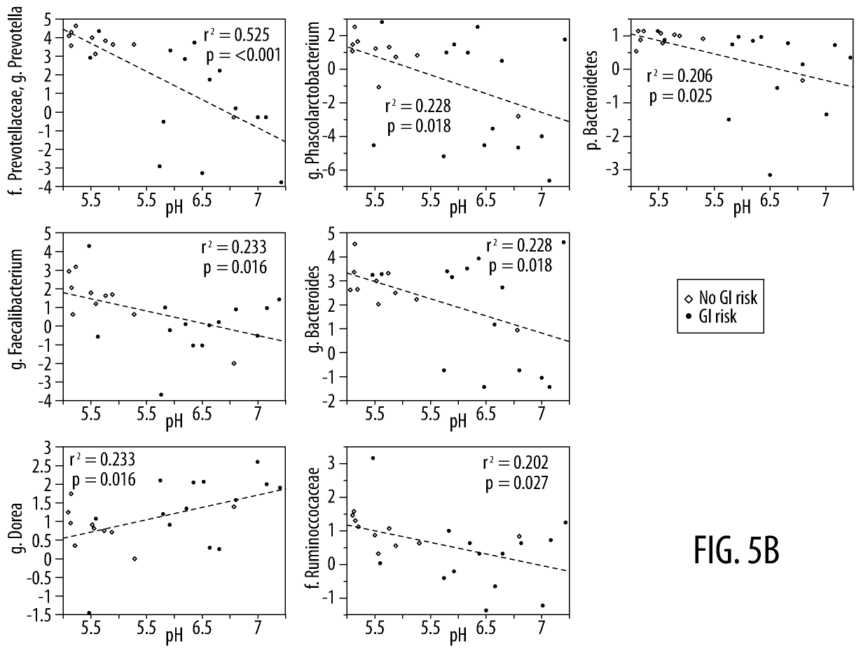

Further this model shows sensitivity 0.916 and specificity 0.75 for detecting an individual having “GI risk” condition and “No GI risk” condition, respectively. Also, PCA was performed using the 16 biomarkers identified by PLSDA model and showed good separation of the “GI risk” vs “No GI risk” population (FIGS. 4A and 4B). Among those 16 biomarkers, the variable “pH” was negatively associated with disease condition (“GI risk” vs “No GI risk”). Correlation analysis of other biomarkers was compared with “pH” and all other biomarkers were significantly correlated either positively or negatively with “pH”. (FIGS. 5A & 5B and Table 3).

TABLE 3
Spearman correlation analysis of response variables against pH

| Variable | by Variable | Spearman p | Prob > Ip I |
|---|---|---|---|
| C2, ppm | pH | −0.802608696 | 2.373E−06 |
| C3, ppm | pH | −0.82 | 9.383E−07 |
| C4, ppm | pH | −0.435652174 | 0.0333432 |
| Total SCFA | pH | −0.815652174 | 1.194E−06 |
| f_Peptococcacede | pH | 0.446956522 | 0.0285463 |
| f_Peptostreptococcaceae | pH | 0.466956522 | 0.0214219 |
| f_Prevotellaceae | pH | −0.805217391 | 2.077E−06 |
| f_Prevotellaceae; g_Prevotella | pH | −0.749565217 | 2.487E−05 |
| f_Ruminococcaceae | pH | −0.459130435 | 0.0240151 |
| f_Ruminococcaceae; g_{—} | pH | −0.486956522 | 0.0158101 |
| g_Dorea | pH | 0.42173913 | 0.0401037 |
| g_Faecalibacterium | pH | −0.539130435 | 0.006557 |
| g_Peptococcus | pH | 0.43826087 | 0.0321831 |
| g_Phascolarctobacterium | pH | −0.428695652 | 0.0366 |
| Evenness | pH | −0.406086957 | 0.0489534 |
| Moisture | pH | 0.8 | 2.706E−06 |
| p_Bacteroidetes | pH | −0.633043478 | 0.0008997 |
| p_Fusobacteria | pH | 0.586086957 | 0.002616 |

### Example 2: Feces Sample Collection

A feces score 5 is good (firm) and feces score 1 is bad (watery). For each fecal score grade 1-5, five fecal samples were collected from individual dogs. Fecal samples were collected immediately after defecation (within 30 minutes) and whole feces were transferred into labeled collection bag for homogenization. The samples were thoroughly homogenized and aliquoted into labeled cryovials and snap frozen into liquid nitrogen. Further, the snap frozen tubes were transferred into −70° C. until further processing. The sample numbers and associated metadata are listed in Table 2.

### Example 3: Measurement of Moisture, pH and Short-Chain Fatty Acids (SCFA)

Fecal pH was measured using a digital pH meter (Hach Model H160 pH Meter) with a pH probe (ISFET Series H Metal Probe), designed for measuring pH in meats. The pH meter is calibrated with each set of samples at three points (pH 4.0, pH 7.0 and pH 10.0).

Fecal SCFAs were measured by using liquid-liquid extraction and Gas Chromatography with flame Ionization detection to quantify specific carboxylic acids in fecal matter. Briefly, fecal samples were measured into centrifuge tubes with an internal standard, and then extracted using an aqueous buffer. An aliquot of that extract was acidified and then extracted using an organic solvent. The organic extract was separated from the aqueous phase, and analyzed using gas chromatography.
LAB-RES-082.0: pH of stool
LAB-RES-45.2: Measurement of Volatile Carboxylic Acids in fecal matter

### Example 4: 16S rRNA Amplicon Sequencing

Total fecal DNA was extracted from frozen fecal samples by using a MoBio PowerFecal DNA extraction kit. Following total DNA extraction, a 16S rDNA amplicon was developed from the samples by employing PCR using the primer sets spanning V3 and V4 hypervariable regions and the amplicons were subsequently analyzed qualitatively by an Agilent 2100 Bioanalyzer. Index PCR was performed, followed by library quantification, normalization and pooling the samples by following manufacturer's instructions. The final pooled sample library was loaded in a Miseq v3 sample loading cartridge kit and the cartridge was placed in a MiSeq (Illumina) sequencer for sequencing the amplicons. The sample sequences were demultiplexed by using MiSeq's in-built “metagenomics” workflow to obtain FASTQ files. FASTQ files were processed by employing QIIME software to classify the sequence reads using Greengenes database to produce the abundance of genera.

Total DNA was extracted from frozen feces samples by using PowerFecal DNA isolation kit (MoBio, Carlsbad, CA) according to the manufacturer's instructions with modification by introducing sonication step before placing the bead tubes with feces sample horizontally for vortexing 15 minutes. PCR amplification was performed by using the primer pairs 347F and 803R spanning the V3-V4 hypervariable regions of 16s rRNA gene along with Illumina adapters (Nossa et al 2013) with following conditions: 25 cycles of 95° C. for 30 s, 55° C. for 30 s, and 72° C. for 45 s, followed by 72° C. for 5 min, in Biorad C1000 Touch thermal cycler. The amplicon was purified by using Agencourt AmPure XP beads (Beckman Coulter) followed by manufacturer's instructions and concentration was determined by employing Qubit fluorometer 3.0 (Life Technologies) with a high sensitivity DNA kit. Further, the amplicon quality was determined by using an Agilent 2100 Bioanalyzer. Index PCR, library quantification, normalization and pooling was performed followed by Illumina's 16S metagenomic sequencing library preparation protocol (Part #15044223 Rev. A). Libraries were mixed with Illumina generated PhiX control library (final concentration 10%) and denatured using fresh NaOH. Final pooled denatured library (6 pm) was loaded in Miseq v3 reagent cartridge kit and performed paired-end sequencing by 2/251 cycles with Miseq Control Software (MCS) 2.4., RTA 1.18.54 and Miseq Reporter 2.4. For every Miseq run, one mock community sample was included as positive control and water was used as a negative control. Both positive and negative control samples were processed identically as fecal DNA samples for the whole sequence processing protocol. LAB-LS-060.1: DNA isolation from bacteria in dog/feline feces-MoBio PowerFecal kit.

### Example 5: Data Analysis

All the identified operational taxonomic units (OTUs) in the samples were included to calculate the Shannon diversity (H) index. Those with less than 75% ubiquity were removed from the data analysis. For the remaining 46 genera (Table 4), the zero counts were imputed, and then all the counts were converted to relative abundances by dividing the individual counts by the total number of counts in the sample. The relative abundances were transformed to centered log ratios (CLRs) for further statistical analysis. A similar approach was followed for the microbiome data at phylum and family level. The R package, ALDEx2, was used for the imputation and transformation.

Partial least squares-discriminant analysis (PLS-DA) was performed to classify the animals to “GI risk” or “No GI risk” using the microbiome CLRs, SCFAs, pH and associated metadata. XLSTAT was used for the PLS-DA analysis.

TABLE 4
Taxonomy of Bacteria Included in the Model

| | |
|---|---|
| g_Bifidobacterium | g_Blautia |
| f_Coriobacteriaceae; g | g_Coprococcus |
| g_Adlercreutzia | g_Dorea |
| g_Collinsella | g_Roseburia |
| g_Slackia | f_Lachnospiraceae; Other |
| f_[Paraprevotellaceae]; g | g_Peptococcus |
| f_[Paraprevotellaceae]; g_[Prevotella] | f_Peptostreptococcaceae; g |
| g_Bacteroides | g_Peptostreptococcus |
| f_Prevotellaceae; g_Prevotella | f_Ruminococcacede; g |
| f_S24-7;g | g_Faecalibacterium |
| g_Lactobacillus | g_Dialister |
| g_Streptococcus | g_Megamonas |
| g_Turicibacter | g_Phascolarctobacterium |
| O_Clostridiales; f_; g | o_Clostridiales; Other; Other |
| f_[Mogibacteriaceae]; g | f_Erysipelotrichaceae; g |
| f_Clostridiaceae; g | g_Allobaculum |
| g_Clostridium | g_Catenibacterium |
| g_SMB53 | g_Fusobacterium |
| f_Clostridiaceae; Other | ; g_Sutterella |
| g_Pseudoramibacter Eubacterium | f_Succinivibrionaceae; g |
| f_Lachnospiraceae; g | g_Anaerobiospirillum |
| f_Lachnospiraceae; g_[Ruminococcus] | f_Enterobacteriaceae; g |
| f_Erysipelotrichaceae; g_[Eubacterium] | Unassigned |

While the invention has been described with respect to specific examples including presently preferred modes of carrying out the invention, those skilled in the art will appreciate that there are numerous variations and permutations of the above-described method and system.

It is to be understood that other embodiments may be utilized and structural and functional modifications may be made without departing from the scope of the present invention. Thus, the spirit and scope of the invention should be construed broadly as set forth in the appended claims.

What is claimed is:

1. A method for identifying a subject dog as being at risk or not at risk for gastrointestinal (GI) disease comprising:
(a) analyzing a fecal sample from the subject dog to determine fecal sample values for one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes; and
(b) comparing the values obtained from (a) with control values from one or more not at risk for GI disease dog for a respective one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes;
wherein the subject dog is not at risk for GI disease when one or more of the fecal sample values are similar or equal to the control values, or wherein the subject dog is at risk for GI disease when one or more of the fecal sample values is different than the control values.

2. The method of claim 1, the (a) analyzing is to determine the fecal sample values for each of the (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes; and
the (b) comparing is with the control values for each respective one of the (i) total short chain fatty acid content (SCFA), (ii) level of C2 SCFA, (iii) level of C3 SCFA, (iv) level of C4 SCFA, (v) pH, (vi) moisture, (vii) fecal score, and (vii) abundance of bacteria of Prevotellaceae, Ruminococcaceae, Bacteroidaceae, Enterobacteriaceae, and Bacteroidetes.

3. The method of claim 2, wherein the subject dog is not at risk for GI disease when the fecal sample has, compared to the control values:
(i) an approximately equal to or higher level total short chain fatty acid content;
(ii) an approximately equal to or higher level of C2 SCFA;
(iii) an approximately equal to or higher level of C3 SCFA;
(iv) an approximately equal to or higher level of C4 SCFA;
(v) an approximately equal to or lower pH;
(vi) an approximately equal to or higher fecal score; and
(vii) an approximately equal to or higher abundance of bacteria of Prevotellaceae, Ruminococcaceae, and Bacteroidaceae, and an approximately equal to or lower abundance of bacteria of Enterobacteriaceae and Bacteriodetes other than Prevotellaceae and Bacteroidaceae.

4. The method of claim 2, wherein the subject dog is at risk for GI disease when the feces sample has, compared to the control values:
(i) a lower level of total short chain fatty acid content;
(ii) a lower level of C2 SCFA;
(iii) a lower level of C3 SCFA;
(iv) a lower level of C4 SCFA;
(v) a higher pH;
(vi) a lower fecal score; and
(vii) a lower abundance of Prevotellaceae, Ruminococcaceae, and Bacteroidaceae, and a higher abundance of bacteria of Enterobacteriaceae and Bacteriodetes other than Prevotellaceae and Bacteroidaceae.

5. The method of claim 1, wherein analyzing the fecal sample for the abundance of bacteria of Prevotellaceae, Ruminococcaceae, Bacteroidaceae, Enterobacteriaceae, and Bacteroidetes comprises amplifying 16S rDNA specific to species within each of Prevotellaceae, Ruminococcaceae, Bacteroidaceae, Enterobacteriaceae, and Bacteroidetes.

6. A kit for identifying a subject dog as being at risk or not at risk for gastrointestinal (GI) disease by obtaining fecal analysis results, the kit comprising at least one of: a reagent or device for determining pH of a fecal sample, one or more reagent or device for measuring volatile carboxylic acids in a fecal sample, and one or more reagent or device for determining an abundance in a fecal sample bacteria of one or more of Prevotellaceae, Ruminococcaceae, Bacteroidaceae, Enterobacteriaceae, and Bacteroidetes, wherein implementing the foregoing produces the results.

7. The kit of claim 6 comprising each of the reagent or device for determining pH of a fecal sample, the one or more reagent or device for measuring volatile carboxylic acids in a fecal sample, and the one or more reagent or device for determining an abundance in a fecal sample for bacteria of each of Prevotellaceae, Ruminococcaceae, Bacteroidaceae, Enterobacteriaceae, and Bacteroidetes.

8. The kit of claim 6, wherein the one or more reagent or device for determining an abundance in a fecal sample of one or more of Prevotellaceae, Ruminococcaceae, Bacteroidaceae, Enterobacteriaceae, and Bacteroidetes comprise PCR primers for amplifying 16S rDNA from target bacteria in Prevotellaceae, Ruminococcaceae, Bacteroidaceae, Enterobacteriaceae, and Bacteroidetes.

9. A method for preventing or treating GI disease comprising identifying whether a subject dog is at risk or not at risk for gastrointestinal (GI) disease by the method of claim 1, and providing to the subject dog a GI disease-mitigating diet when the identifying step is a conclusion that the subject dog is at risk.

10. The method of claim 9, wherein the subject dog is not at risk for GI disease when the fecal sample has, compared to the control values:
(i) an approximately equal to or higher level total short chain fatty acid content;
(ii) an approximately equal to or higher level of C2 SCFA;
(iii) an approximately equal to or higher level of C3 SCFA;
(iv) an approximately equal to or higher level of C4 SCFA;
(v) an approximately equal to or lower pH;
(vi) an approximately equal to or higher fecal score; and
(vii) an approximately equal to or higher abundance of bacteria of Prevotellaceae, Ruminococcaceae, and Bacteroidaceae, and an approximately equal to or lower abundance of bacteria of Enterobacteriaceae and Bacteriodetes other than Prevotellaceae and Bacteroidaceae.

11. The method of claim 9, wherein the subject dog is at risk for GI disease when the feces sample has, compared to the control values:
(i) a lower level of total short chain fatty acid content;
(ii) a lower level of C2 SCFA;
(iii) a lower level of C3 SCFA;
(iv) a lower level of C4 SCFA;
(v) a higher pH;
(vi) a lower fecal score; and
(vii) a lower abundance of bacteria of Prevotellaceae, Ruminococcaceae, and Bacteroidaceae, and an approximately higher abundance of bacteria of Enterobacteriaceae and Bacteriodetes other than Prevotellaceae and Bacteroidaceae.

12. The method according to claim 9, wherein the GI disease-mitigating diet comprises a dog food composition comprising:
from about 1 to about 10 wt. % of quinoa;
from about 1 to about 10 wt. % of amaranth;
from about 0.5 to about 8 wt. % of egg shell membrane; and
from about 2 to about 20 wt. % of dietary fiber, the dietary fiber comprising:
i) from about 1 to about 18 wt. % of insoluble fiber, and
ii) from about 0.5 to about 8 wt. % of soluble fiber
wherein all weight percentages are based on the total weight of the dog food composition on a dry matter basis.

13. The method according to claim 12, wherein the dog food composition further comprises from about 0.1 to about 10 wt. % of fatty acids.

14. The method according to claim 13, wherein the fatty acids comprise a polyunsaturated fatty acid.

15. The method according to claim 14, wherein the polyunsaturated fatty acid comprises an omega-3 fatty acid, an omega-6 fatty acid, linolenic acid, stearidonic acid, eicosatetraenoic acid, eicosapentaenoic acid, docosapentaenoic acid, docosahexaenoic acid, calendic acid, eicosadienoic acid, arachidonic acid, docosadienoic acid, adrenic acid, osbond acid, tetracosatetraenoic acid, or tetracosapentaenoic acid, or a combination of two or more thereof.

16. A device comprising an input, a memory, a processor, and an output, wherein
the input is configured to receive the results of a method for identifying a subject dog as being at risk or not at risk for gastrointestinal (GI) disease;
the memory comprises processor executable instructions for comparing the results to control values, and for preparing a conclusion of at risk or not at risk based on the comparing, and outputting the results;
the processor is configured to execute the processor executable instructions; and
the output is configured to display the conclusions.

17. The device of claim 16, wherein the processor executable instructions further comprise instructions to display a recommendation to feed to the subject a GI disease-mitigating diet when conclusion is that the subject dog is at risk, the processor is further configured to execute the instructions to display a recommendation, and the output is further configured to display the recommendation.

18. The device of claim 17, wherein the recommendation to feed to the subject a GI disease-mitigating diet is to feed to the subject a dog food composition comprising:
from about 1 to about 10 wt. % of quinoa;
from about 1 to about 10 wt. % of amaranth;
from about 0.5 to about 8 wt. % of egg shell membrane; and
from about 2 to about 20 wt. % of dietary fiber, the dietary fiber comprising:
i) from about 1 to about 18 wt. % of insoluble fiber, and
ii) from about 0.5 to about 8 wt. % of soluble fiber wherein all weight percentages are based on the total weight of the dog food composition on a dry matter basis.

19. The device of claim 18, wherein the recommendation to feed to the subject a GI disease-mitigating diet is to feed to the subject a dog food composition further comprises from about 0.1 to about 10 wt. % of fatty acids, and the fatty acids comprise an omega-3 fatty acid, an omega-6 fatty acid, linolenic acid, stearidonic acid, eicosatetraenoic acid, eicosapentaenoic acid, docosapentaenoic acid, docosahexaenoic acid, calendic acid, eicosadienoic acid, arachidonic acid, docosadienoic acid, adrenic acid, osbond acid, tetracosatetraenoic acid, or tetracosapentaenoic acid, or a combination of two or more thereof.

20. The device of claim 16, wherein the method comprises:
(a) analyzing a fecal sample from the subject dog to determine fecal sample values for one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes; and
(b) comparing the values obtained from (a) with control values from one or more not at risk for GI disease dog for a respective one or more of (i) total short chain fatty acid content (SCFA), (ii) level of C2 SCFA, (iii) level of C3 SCFA, (iv) level of C4 SCFA, (v) pH, (vi) moisture, (vii) fecal score, and (viii) abundance of bacteria of Prevotellaceae, Ruminococcaceae, Bacteroidaceae, Enterobacteriaceae, and Bacteroidetes;
wherein the subject dog is not at risk for GI disease when one or more of the fecal sample values are similar or equal to the control values, or wherein the subject dog is at risk for GI disease when one or more of the fecal sample values is different than the control values.
